(19) United States
(12) Patent Application Publication
(10) Pub. No.: US 2023/0067015 A1
(43) Pub. Date: Mar. 2, 2023

(54) SYSTEMS AND METHODS FOR ANTIBACTERIAL SUSCEPTIBILITY TESTING USING DYNAMIC LASER SPECKLE IMAGING

(71) Applicant: The Penn State Research Foundation, University Park, PA (US)

(72) Inventors: Seyedehaida EBRAHIMI, University Park, PA (US); Chen ZHOU, University Park, PA (US); Jasna KOVAC, University Park, PA (US); Zhiwen LIU, University Park, PA (US); Keren ZHOU, University Park, PA (US)

(21) Appl. No.: 17/759,995

(22) PCT Filed: Feb. 3, 2021
(86) PCT No.: PCT/US2021/016471

§ 371 Date: Aug. 2, 2022

Related U.S. Application Data
(60) Provisional application No. 62/969,363, filed on Feb. 3, 2020.

(51) Int. Cl. C12Q 1/18; G01N 21/47

(52) U.S. Cl. CPC C12Q 1/18; G01N 21/4788; G01N 2021/479

(57) ABSTRACT

A method for antibacterial susceptibility testing includes preparing a set of two or more samples of a plurality of bacterial cells from a patient; adding a different amount of a selected drug to each sample; illuminating at least a portion of a sample using a coherent illumination source; capturing a series of speckle images over time of at least a portion of the illuminated sample; and determining an inhibition status of the sample using a machine-learning classifier applied to the series of speckle images. The steps of illuminating the sample, capturing a series of images, and determining an inhibition status are repeated for each sample of the set of two or more samples. The method may include transforming the series of speckle images to a frequency series of speckle images; and determining the inhibition status of the sample uses the machine-learning classifier applied to the frequency series of speckle images.

### CROSS-REFERENCE TO RELATED APPLICATIONS

This application claims priority to U.S. Provisional Application No. 62/969,363, filed on Feb. 3, 2020, now pending, the disclosure of which is incorporated herein by reference.

### STATEMENT REGARDING FEDERALLY SPONSORED RESEARCH

This invention was made with government support under Grant No. DMR1420620 awarded by the National Science Foundation and under Hatch Act Project No. PEN04646 awarded by the United States Department of Agriculture. The Government has certain rights in the invention.

### BACKGROUND OF THE DISCLOSURE

Antimicrobial resistance (AMR) is among the most serious health threats of all time. Antimicrobial resistant pathogens cause an estimated 2.8 million infections and 35,000 deaths per year in the United States. The fatality rate due to antimicrobial resistant infections is expected to reach 10 million per year by 2050 if the expansion of AMR is not effectively mitigated. Misuse and overuse of broad-spectrum antibiotics due to the absence of reliable and accurate rapid antibacterial susceptibility testing (RAST) is contributing to the spread of AMR infections. Gold standard AST techniques, including disk diffusion and broth microdilution (BMD), take over 16 hours to complete which limits their utility in cases of severe sepsis. The EUCAST developed a RAST method in 2019 that can determine the MIC results of 7 species within 4˜8 hours based on the disk diffusion method. Further development of accurate RAST that produces results concordant with gold standard methods is therefore critically needed to speed up AST to support data-informed antibiotic prescription and improve patient treatment outcomes.

A series of molecular and phenotypic technologies have recently been developed for RAST and detection of AMR. Molecular methods rely on the detection of target nucleic acid sequences markers to determine and predict the antibiotic resistance. Amplification of target DNA using polymerase chain reaction (PCR), isothermal recombinase polymerase amplification (RPA), or loop-mediated isothermal amplification (LAMP), combined with fluorescence detection can produce the results of a single assay within an hour. Due to diverse genetic resistance determinants that do not have sufficient correlation with resistance phenotypes, culture-independent AST testing cannot be broadly applied to all pathogens without extensive prior knowledge of their biology and underlying genetics. Furthermore, molecular methods still fail to detect resistance in cases where novel resistance mechanisms have not yet been characterized.

Compared to molecular methods, phenotype-based ASTs in many cases provide information that is more relevant to clinical outcomes. Among different phenotypic methods, Accelerate Pheno (Accelerate Diagnostics, Inc., Tucson, Ariz., USA) is currently the only FDA-approved RAST technique. This method measures morphokinetic cellular changes that occur due to exposure to antibiotics. Accelerate Pheno combines fluorescence in situ hybridization (FISH) and automated microscopic imaging to identify bacteria. Baltekin et al. used single-cell imaging in a microfluidic cartridge with phase-contrast microscopy and identified resistant bacteria within 30 mins. A method reported by Schoepp et al. has further improved the time-to-results by determining the antimicrobial susceptibility of E. coli directly from clinical urine samples in 30 min. Their technique, coupled with digital LAMP, measures microbial growth based on quantification of nucleic acids using quantitative PCR. One of the limitations of this ultra-fast technique is assuming that slowed or halted DNA replication after 15 min antibiotic exposure indicates susceptibility of a bacterial population to a given antibiotic. However, the bacterial population response within the first 15 min is typically highly variable among assay replicates, which can increase error in the prediction of susceptibility, especially when borderline MIC concentrations of antibiotics are tested.

Change of bacterial viability in response to antibiotics can also be measured by probing bacterial shape, motion, size/mass, or respiration/redox state. Among them, AST methods which rely on monitoring bacterial motion have been extensively explored. Johnson et al. used the phase noise of a resonant crystal to show that the motion of E. coli was attenuated after treatment with polymyxin B or ampicillin. This work demonstrates bacterial motion as a promising characteristic for RAST. Yu et al. reported a phenotypic AST that utilized deep learning to analyze freely moving E. coli cells in urine to determine the minimum inhibitory concentration (MIC) in 30 mins. In another RAST method developed by Resistell Co., change of bacterial viability is measured using a resonating cantilever (as used in atomic force microcopy) which is sensitive to bacterial movement. However, these methods have poor sensitivity and/or require labeling or indicative markers such as redox-active chemicals, which can interfere with cellular physiology. In addition, some of these methods still need advanced imaging/analysis setups, such as high resolution optical microscopy, atomic force microcopy, or complex optical setups, which limits their broad applicability in clinical diagnostics.

Compared to optical methods that visually assess changes in the shape, length, and/or motion of bacterial cells, measuring the light attenuation scattered off bacterial populations is a simpler method that eliminates the need for bulky and high-cost optical setup. One of such methods is laser scattering, which has been utilized in developing RAST. For example, the BacterioScan system (BacterioScan Inc.) measures the optical density (OD) of a liquid sample as well as the low-angle laser scattered intensity which enables measurements of significantly lower OD levels compared to traditional ratiometric transmittance measurements. However, this method is based solely on OD, which does not provide substantial improvement of the RAST turnaround time (˜6 hrs).

### BRIEF SUMMARY OF THE DISCLOSURE

In a first aspect, the present disclosure provides a method for antibacterial susceptibility testing of a sample. The method includes preparing a set of two or more samples, each sample including a plurality of bacterial cells from a patient; adding a different amount of a selected drug to each sample of the set of two or more samples; illuminating at least a portion of a sample of the set of two or more samples using a coherent illumination source; capturing a series of speckle images over time of at least a portion of the illuminated sample; determining an inhibition status of the sample using a machine-learning classifier applied to the series of speckle images; and repeating the steps of illuminating at least a portion of the sample, capturing a series of speckle images over time, and determining an inhibition status of the sample, for each remaining sample of the set of two or more samples. Each series of speckle images may be captured over any suitable measurement period, such as a measurement period of between 5-20 seconds, inclusive, (or shorter or longer measurement periods), such as, for example, a measurement period of 10 seconds. The set of samples may include two or more samples, and a minimum inhibitory concentration (MIC) of the selected drug is determined based on the inhibition status of each sample.

In some embodiments, the method includes transforming the series of speckle images to a frequency series of speckle images; and wherein determining the inhibition status of the sample uses the machine-learning classifier applied to the frequency series of speckle images. The machine-learning classifier may be an artificial neural network having a number of neurons in an input layer corresponding to a number of frequency components in the frequency series of speckle images. The inhibition status of the sample may be determined using the machine-learning classifier by classifying each pixel of the frequency series of speckle images as either inhibited or not inhibited using the machine-learning classifier; calculating a percentage of pixels classified as inhibited; and determining an inhibition status of inhibited when the percentage of pixels classified as inhibited is greater than 50%, and an inhibition status of not-inhibited when the percentage of pixels classified as inhibited is less than or equal to 50%. In some embodiments, the method includes normalizing the frequency series of speckle images such that a DC term is normalized to 1.

In some embodiments of the method, the amounts of the selected drug added to each sample of the set of two or more samples yields minimum inhibitory concentrations (MIC).

The series of speckle images over time may be captured at multiple time points after adding the selected drug to the samples. For example, in some embodiments, the series of speckle images over time is captured at a time at least 60 minutes after adding the selected drug to the samples.

In some embodiments, the series of speckle images is captured in an angular range defined by a Mie scattering model, between an optical axis of the image sensor and an optical axis of the illumination source. In some embodiments, the method includes determining an average bacterium size of each sample using the Mie scattering model fitted with the series of speckle images. In some embodiments, the method further includes determining an average intensity value of each series of speckle images.

In another aspect, the present disclosure provides a system for antibacterial susceptibility testing of a sample. The system includes a sample holder; a coherent illumination source configured to illuminate at least a portion of a sample within the sample holder; an image sensor (e.g., a camera of a smartphone, or other sensor) positioned to receive light scattered by the sample thereby creating a series of speckle images over time; and a processor. The processor is configured to perform any of the methods disclosed herein. For example, the processor may be configured to receive from the image sensor the time series of speckle images; determine an inhibition status of the sample using a machine-learning classifier applied to the series of speckle images over time. The series of speckle images may be captured over any suitable measurement period, such as a measurement period of between 5-20 seconds, inclusive, (or shorter or longer measurement periods), such as, for example, a measurement period of 10 seconds.

In some embodiments, the processor is further configured to transform the series of speckle images over time into a frequency series of speckle images, and wherein determining the inhibition status of the sample uses the machine-learning classifier applied to the frequency series of speckle images. For example, in some embodiments, the processor is configured to determine an inhibition status of the test sample using the machine-learning classifier by classifying each pixel of the frequency series of images as either inhibited or not inhibited using the machine-learning classifier; calculating a percentage of pixels classified as inhibited; and determining an inhibition status of inhibited when the percentage of pixels classified as inhibited is greater than 50%, and an inhibition status of not-inhibited when the percentage of pixels classified as inhibited is less than or equal to 50%. In some embodiments, the frequency series of speckle images is normalized such that a DC term is normalized to 1. The machine-learning classifier may be an artificial neural network having a number of neurons in an input layer corresponding to a number of frequency components in the frequency series of speckle images.

In some embodiments, an optical axis of the image sensor is positioned in an angular range with respect to an optical axis of the illumination source defined by a Mie scattering model. The processor may be further configured to determine an average bacterium size of the sample using the Mie scattering model fitted with the series of speckle images.

In some embodiments, the processor is further configured to resize the series of speckle images. In some embodiments, the processor is further configured to determine an average intensity value of the series of speckle images.

### DESCRIPTION OF THE DRAWINGS

For a fuller understanding of the nature and objects of the disclosure, reference should be made to the following detailed description taken in conjunction with the accompanying drawings, in which:

FIG. 1. Diagram of an experimental embodiment of the present dynamic speckle imaging for rapid AST (DyRAST) process. After expansion by the lens, the laser beam is scattered by the bacterial culture in the cuvette containing different concentration of antibiotic (ρ_{Antb}). At each time point (tᵢ), the dynamic speckle patterns were recorded, for total time of 10 sec, 500 frames. After pre-processing, Fourier analysis revealed the information about bacterial motion. The Fourier results were then fed into an artificial neural network (ANN). The trained ANN model could accurately predict the minimum inhibitory concentration (MIC) in 60 min.

FIG. 2. Raw representative speckle images of E. coli K-12 exposed to ampicillin (AMP) and gentamicin (GEN) at different concentrations (ρ). MIC of ampicillin and gentamicin was 4 μg/mL and 2 μg/mL, respectively. The images were collected over a total period of 2 hrs with 30 min intervals at time points, tᵢ (i=0, 1, . . . , 5). The images were collected using an optimized setup (Setting #3).

FIG. 3. (a) The OD₆₀₀ values vs. treatment time and (b) the average intensity results obtained from raw dynamic laser speckle images of E. coli K-12 with ampicillin with ρ_{AMP}=0, 1, 2, 4, and 8 μg/mL. (c) and (d): The corresponding results for gentamicin with ρ_{GNT}=0, 0.5, 1, 2 and 4 μg/mL. The dashed lines in part (a) and (d) indicate the detection limit of the commercial OD measurement system. The MIC for ampicillin and gentamicin were 4 μg/mL and 2 μg/mL, respectively.

FIG. 4. A diagram depicting an exemplary data preprocessing and machine learning algorithm for prediction of the minimum inhibitory concentration (MIC) and whether a particular antibacterial treatment is inhibitory or not. (a) Raw speckle images were collected at each tᵢ. The incident laser beam was blocked to ensure it does not directly hit the camera to avoid pixel saturation. The time series at each pixel was Fourier transformed with the DC term normalized to 1. Fourier results, i.e., the resultant features, were fed into the ANN model, with 249 neurons as the input, 300 hidden units, and 2 output neurons for binary classification. (b) The trained ANN model was tested using a separate set of data. Similarly, 20,000 pixel-level spectra were preprocessed and fed into the trained ANN. Through voting (threshold at 50%), we determined the susceptibility of the bacterial sample (“Inhibited” vs. “Non-Inhibited”). (c) Comparison of the ANN algorithm with different resizing factor. Prediction accuracy of ANN with 100×200, 25×50, or 10×20 resizing factor is almost identical (within 5% difference).

FIG. 5. (a) Time-kill curves for E. coli K-12 with different concentrations of ampicillin (ρ_{AMP}). The prediction accuracy of machine learning algorithm improves with time: (b) the results using DLSI data collected at t₂=30 min, and (c) the results using DLSI data collected at t₃=60 min. After 60 min, the method can identify MIC with 100% accuracy. The dashed lines indicate the voting threshold (50%) to predict AST results with 100% accuracy, i.e., “Inhibited” for 1×MIC and 2×MIC, while at the same time, “Non-Inhibited” for 0.25×MIC and 0.5×MIC. Ampicillin MIC was 4 μg/mL.

FIG. 6. (a) Time-kill curves for E. coli K-12 with different concentrations of gentamicin (ρ_{GEN}). The prediction accuracy of machine learning algorithm improves with time: (b) the results using DLSI data collected at t₂=30 min, and (c) the results using DLSI data collected at t₃=60 min. Interestingly, the method can predict MIC for gentamicin in 30 min (compared to 60 min for AMP). The dashed lines indicate the voting threshold (50%) to predict AST results with 100% accuracy, i.e., “Inhibited” for 1×MIC and 2×MIC, while at the same time, “Non-Inhibited” for 0.25×MIC and 0.5×MIC. Gentamicin MIC was 2 μg/mL.

FIG. 7. Voting strategy enables accurate and rapid prediction of ceftriaxone's MIC. (A) Time-kill curves for E. coli K-12 with different concentrations of ceftriaxone. The prediction percentage of machine learning algorithm improves with time: (B) results using DLSI data collected at t₁=60 min and (C) results using DLSI data collected at t₄=240 min. The method can predict MIC for ceftriaxone in 240 min. The dashed lines indicate the voting threshold (50%) to predict AST results with high accuracy, i.e., “inhibited” for 1×MIC, 2×MIC, and 4×MIC, while at the same time “non-inhibited” for 0.25×MIC. Ceftriaxone MIC was 0.0625 μg/mL.

FIG. 8. Photograph of the test setup with optical modules position parameters.

FIG. 9. Raw speckle images obtained using Setting #1. In this case, the scattered light was too weak (consistent with the Mie scattering model discussed below) for pattern recognition by machine learning.

FIG. 10. Raw speckle images obtained using Setting #2. In this case, the signals saturate due to high intensity of the scattered light.

FIG. 11. Raw speckle images obtained using Setting #3. This setting achieved the best results among the tested embodiments based on the machine learning (ML) analysis.

FIG. 12. (a) The location of captured window of camera relative to the laser beam. A typical speckle pattern has 1,000 pixels in the lateral direction, and 2,000 in the vertical direction. (b) A representative speckle image. The arrow indicates the lateral direction. (c) The simulated result using Mie scattering model, assuming n(E. coli)=1.384, n(water)=1.33, particle size=0.5 μm, and laser wavelength=633 nm. The angular dependence of the intensity is shown. The direct ray direction (0 degree) has the most scattering light, and it approaches to 0 around 40 degree. In the experiment, the lateral angle between 11 degrees to 22 degrees was collected, which is shown in the solid line plot. It is around 28% of the scattering light across the lateral direction in total.

FIG. 13. A representative intensity plot along the lateral direction. To fit the Mie scattering model, we calculated the ratio of 1,000^{th} pixel to the 1^{st} pixel as the fitting parameter to estimate the particle size.

FIG. 14. The FT result of ampicillin for E. coli K12 for three independent experiments at time points of 30 min, 1 h, 90 min, and 2 h. The rows correspond to different experiments and the columns indicate different time points.

FIG. 15. The FT result of the gentamicin for E. coli K12 for three independent experiments at time points of 30 min, 1 h, 90 min, and 2 h. The rows correspond to different experiments and the columns indicate different time points.

FIG. 16. The FT result of preliminary studies for (A) MDR E. coli PS00278A with different concentrations of ampicillin, ρ_{AMP}=0 (control), 16 μg/mL (G1), and 2 mg/mL (G2)), (B) MDR E. coli PS00278A with different concentrations of gentamicin with ρ_{GNT}=0, 8 μg/mL (G1), and 128 μg/mL (G3) and (C) S. aureus PS00975A with different concentrations of ampicillin, ρ_{AMP}=0 (G1) and 4 μg/mL (G4) at immediate, 30 min, 1 hour, 90 min, and 2 hours.

FIG. 17. The FT result of preliminary studies of E. coli strain K12 with different concentrations of ceftriaxone, p_{CFT}=0 (GS), 0.016 μg/mL (G1), 0.0625 μg/mL (G2), and 0.125 μg/mL (G3) at immediate, 1 hour, 2 hours, 3 hours, and 4 hours.

FIG. 18. Confusion matrix for ampicillin data at (a) t₁, (b) t₂ ,(c) t₃, (d) t₄ at the training process before testing on an independet experiments.

FIG. 19. Confusion matrix for gentamicin at (a) t₁, (b) t₂ ,(c) t₃, (d) t₄ at the training process before testing on the independet experiments.

FIG. 20. The machine learning prediction results for E. coli K-12 inhibition by ampicillin. Using t₁=30 min data, t₂=60 min, t₃=90 min, and t₄=120 min data. After 60 min, the method can identify MIC with confidence.

FIG. 21. The machine learning prediction results for E. coli K-12 inhibition by gentamicin. Using t₁=30 min data, t₂=60 min, t₃=90 min, and t₄=120 min data. After 60 min, the method can identify MIC with confidence.

FIG. 22. (A) The OD₆₀₀ values vs. treatment time, (B) the raw dynamic laser speckle images at 0, 1, and 2 hours, and (C) the average intensity results of E. coli K12 with ρ_{CFT}=0 and 0.0625 μg/mL (MIC).

FIG. 23. The ANN prediction results using DLSI data collected at t₂=120 min, and (B) the results using DLSI data collected at t₃=180 min.

FIG. 24. The time-kill curves for (A) MDR E. coli PS00278A with ampicillin, (B) MDR E. coli PS00278A with gentamicin, and (C) clinical S. aureus PS00975A with ampicillin.

FIG. 25. The OD₆₀₀ values vs. treatment time, (B) the average intensity results, and (C) the raw dynamic laser speckle images at 0, 1, and 2 hours of MDR E. coli PS00278A with ρ_{AMP}=0, 16 μg/mL (breakpoint), and 2 mg/mL (MIC).

FIG. 26. (A) The OD₆₀₀ values vs. treatment time (B) the average intensity results, and (C) the raw dynamic laser speckle images at 0, 1, and 2 hours of MDR E. coli PS00278A with ρ_{GNT}=0, 8 (breakpoint), and 128 μg/mL (MIC).

FIG. 27. (A) The OD₆₀₀ values vs. treatment time, (B) the average intensity results, and (C) the raw dynamic laser speckle images at 0, 1, and 2 hours for S. aureus PS00975A with ρ_{AMP}=0 and 4 μg/mL (MIC).

FIG. 28. Time-evolution of the FT values at 10 Hz for: (A) MDR E. coli treated with ampicillin (MIC: 2 mg/mL and breakpoint concentration: 16 μg/mL), (B) MDR E. coli treated with gentamicin (MIC: 128 μg/mL) and breakpoint concentration: 8 μg/mL), (C) S. aureus treated with ampicillin (MIC: 4 μg/mL) and no antibiotic.

FIG. 29. E. faecalis treated with ampicillin (AMP) in 10% urine. (A) The average intensity results obtained from raw dynamic laser speckle images with ρ_{AMP}=0.25, 0.5, 1, and 2 μg/mL. (B) The OD₆₀₀ values vs. treatment time. (C) The machine learning results using DLSI data collected at t₃=60 min.

FIG. 30. E. faecalis treated with imipenem in MHB. (A) The average intensity results obtained from raw dynamic laser speckle images with ρ_{IMP}=0.25, 0.5, 1, and 2 μg/mL. (B) The OD₆₀₀ values vs. treatment time. (C) The machine learning results using DLSI data collected at t₃=60 min.

FIG. 31. The DLSI method can distinguish between E. faecalis and E. coli in 30 minutes.

The results are obtained with cells spiked in urine.

FIG. 32. Comparison of the present disclosure with some of the recent size/motion-based RAST methods.

FIG. 33 is a chart of a method according to an embodiment of the present disclosure.

FIG. 34 is a diagram of a system according to another embodiment of the present disclosure.

### DETAILED DESCRIPTION OF THE DISCLOSURE

Unless defined otherwise herein, all technical and scientific terms used in this disclosure have the same meaning as commonly understood by one of ordinary skill in the art to which this disclosure pertains.

Every numerical range given throughout this specification includes its upper and lower values, as well as every narrower numerical range that falls within it, as if such narrower numerical ranges were all expressly written herein.

This disclosure provides compositions, methods and systems for analyzing microorganisms, including but not necessarily limited to susceptibility to antibiotics. The disclosure includes all steps described herein, alone and in combination, sequentially, and in all possible orders. Any step, component, reagent, etc., may be omitted from the present disclosure. The disclosure includes devices described herein in operation, e.g., during analysis of microorganisms. All reagents, periods of time, temperatures, and all physical and optical values disclosed herein are encompassed by the disclosure. In an embodiment, the disclosure provides for accurately determining AST of bacteria in a sample is not more than 60 minutes.

In embodiments, one or more components of a device or system of this disclosure can be connected to or in communication with a digital processor and/or a computer running software to interpret a signal. A processor may also be included as a component of the device or system comprising the device, wherein the processor runs software or implements an algorithm to interpret a detectable signal, and may generate a machine and/or user readable output. In embodiments, information obtained by the device/system can be monitored in real-time by a computer, and/or by a human operator. In certain embodiments, the disclosure provides as an embodiment or component of the system a non-transitory computer readable storage media for use in performing an algorithm to control signal generation and/or detection, and/or for monitoring and/or recording signaling events. In embodiments, a system described herein may operates in a networked environment using logical connections to one or more remote computers. In embodiments, a result obtained using a device/system/method of this disclosure is fixed in a tangible medium of expression. The result may be communicated to, for example, a health care provider.

All systems, devices, and methods as depicted herein, including all components of such systems and steps of the methods, alone and in all possible combinations, are included in this disclosure. Non-limiting examples of devices and device components are depicted in the figures of this disclosure. Variations on the devices and components will be understood by those skilled in the art, given the benefit of this disclosure.

Any result obtained using the devices, systems and methods of this disclosure can be compared to any suitable reference, such as a known value, or a control sample or control value, suitable examples of which will be apparent to those skilled in the art, given the benefit of this disclosure.

In the present disclosure, we utilize dynamic laser speckle imaging to quantify bacterial micromotion and correlate the decrease of micromotions with the inhibitory effects of antibiotics. The method is based on the phenomenon of laser light scattering in scattering media, such as biological tissues. The self-interference of the scattered light produces a speckle pattern that conveys characteristics of the scatterers and the nature of their dynamic behavior over time. The differentiation between dead and living bacterial cells is achieved based on the analyses of the dynamic laser speckle images. When the light is scattered from a static turbid medium, a constant laser speckle pattern is generated due to the fixed phase. On the contrary, if particles in the medium exhibit movement, the scattering pattern will evolve over time, generating a dynamic speckle pattern. The time series of dynamic speckle images contain information about particles' kinetic behavior and can be used as a means to probe the effect of environmental triggers, including antibiotics, on their motion. For example, Murialdo et al. used dynamic laser speckle imaging (DLSI) to detect different degrees of motility and chemotaxis in bacteria swarming plates. Ramirez-Miquet et al. proposed a technique combining speckle imagining with a digital image information technology to track multiplying E. coli and S. aureus cells deposited on agar plates at high concentrations of 1.5×10⁹ cell/mL and 10⁹ cell/mL, respectively. They compared the mean viability of each pathogen and showed this speckle imaging method has the potential to detect biological activity in 15 mins.

Despite these efforts, to the best of our knowledge, there is no report of an application of time-resolved DLSI for antibacterial susceptibility testing in liquid samples. Moreover, previously reported assays are not sensitive enough for rapid analysis of cells at 5×10⁵ cells/mL, a concentration required by the gold standard protocols. We show that analysis of time-resolved DLSI data using an artificial neural network (ANN) can identify the MIC of ampicillin and gentamicin for E. coli K-12 in 60 minutes with high accuracy compared to gold standard methods using a voting strategy for the ANN predictions. In addition to ampicillin and gentamicin, our method can determine MIC of ceftriaxone accurately. Additional tests were conducted on a multidrug-resistant (MDR) strain of E. coli and a clinical isolate of Staphylococcus aureus (S. aureus). The results were validated using the gold standard broth microdilution. In our machine learning model, we adapted a multilayer perceptron (MLP) for classification and prediction. MLP can approximate any measurable function to any desired degree of accuracy with its capability to set highly nonlinear boundaries between classes, and has proven as a superior algorithm for many applications, such as malware detection system and lung cancer classification. The developed dynamic laser imaging-based rapid antibacterial susceptibility testing method (DyRAST) is fast, label-free, and eliminates the need for advanced microscopy systems. Compared with the method of dynamic light scattering (DLS), which is commonly used to characterize nanoparticles, DLSI can simultaneously capture a time series of laser scattering patterns over a range of angles (limited by the field of view of the image sensor). The large amount of evolving spatiotemporal data lend to the use of advanced data analytics (such as machine learning in this work), which is applicable to both single scattering or multiple scattering regimes and can thus enable analysis of both highly diluted and highly concentrated samples. In addition, development of DyRAST involves simple optics and electronics (with potential to incorporate consumer electronics such as cellphone cameras), which can significantly improve the accessibility of the phenotypic RAST.

In this disclosure, we show that analysis of the time-resolved dynamic laser speckle images (DLSI) using artificial neural network (ANN) was able to identify a MIC of ampicillin and gentamicin for E. coli K-12 in only 60 minutes with 100% accuracy by setting a voting threshold of 50% for the ANN prediction. The predictions were validated using gold standard broth microdilution. DyRAST is a rapid, label-free phenotypic AST technique that utilizes simple optical instrumentation. Dynamic speckle imaging eliminates the need for advanced microscopy systems, which significantly simplifies the RAST. Moreover, the developed method has a potential for adjustment to operate using consumer-level components, such as smartphone camera and off-the-shelf laser diodes.

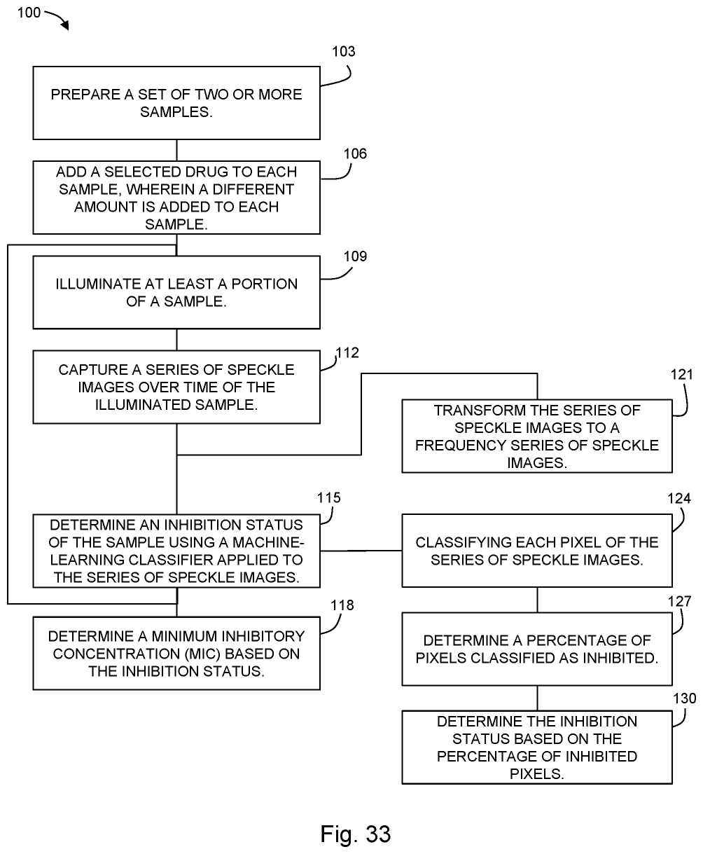

With reference to FIG. 33, the present disclosure may be embodied as a method 100 for antibacterial susceptibility testing of a sample. The method 100 includes preparing 103 a set of two or more samples. Each sample includes a plurality of bacterial cells from a patient. A different amount of a selected drug is added 106 to each sample of the set of two or more samples. The method 100 includes illuminating 109 at least a portion of a sample of the set of two or more samples using a coherent illumination source. The coherent light source may be, for example, a laser. The method 100 includes capturing 112 a series of speckle images over time of at least a portion of the illuminated sample. The series of speckle images may be captured over any suitable measurement period, such as a measurement period of between 5-20 seconds, inclusive, (or shorter or longer measurement periods), such as, for example, a measurement period of 10 seconds. An inhibition status of the sample is determined 115 using a machine-learning (ML) classifier applied to the series of speckle images. The steps are repeated for each remaining sample of the set of two or more samples.

The method may include determining 118 a minimum inhibitory concentration (MIC) of the selected drug based on the inhibition status of each sample.

In some embodiments, the series of speckle images over time is transformed 121 into a frequency series of speckle images. For example, the series of speckle images may be transformed into the frequency domain using Fourier transform (FT). With a frequency series of speckle images, the step of determining 115 the inhibition status of the sample uses the machine-learning classifier applied to the frequency series of speckle images. In some embodiments, the machine-learning classifier is an artificial neural network (ANN) having a number of neurons in an input layer corresponding to a number of frequency components in the frequency series of speckle images. The inhibition status of the sample may be determined 115 using the machine-learning classifier by classifying 124 each pixel of the frequency series of speckle images as either inhibited or not inhibited using the machine-learning classifier. A percentage of pixels classified as inhibited is then determined 127. The inhibition status is determined 130 to be “inhibited” when the percentage of pixels classified as inhibited is greater than 50%, and “not-inhibited” when the percentage of pixels classified as inhibited is less than or equal to 50%. IT should be noted that a threshold of 50% is used herein to illustrate an embodiment and the threshold may be higher or lower (for example, 40%, 45%, 50%, 55%, 60%, 65%, 70%, 80%, or values therebetween). In some embodiments, the method includes normalizing the frequency series of speckle images such that a DC term is normalized to 1.

In some embodiments of the method 100, the amounts of the selected drug added to each sample of the set of two or more samples yields minimum inhibitory concentrations (MIC). In the exemplary embodiments described herein, non-limiting MICs of 0.25×, 0.5×, 1×, 2×, and/or 4× MIC are used to illustrate the embodiments—other MIC values may be used. The series of speckle images over time may be captured at multiple time points after adding the selected drug to the samples. For example, in some embodiments, the series of speckle images over time is captured at a time at least 60 minutes after adding the selected drug to the samples.

In some embodiments of the method, the series of speckle images is captured in an angular range defined by a Mie scattering model, between an optical axis of the image sensor and an optical axis of the illumination source. In some embodiments, the method includes determining an average bacterium size of each sample using the Mie scattering model fitted with the series of speckle images. In some embodiments, the method further includes determining an average intensity value of each series of speckle images.

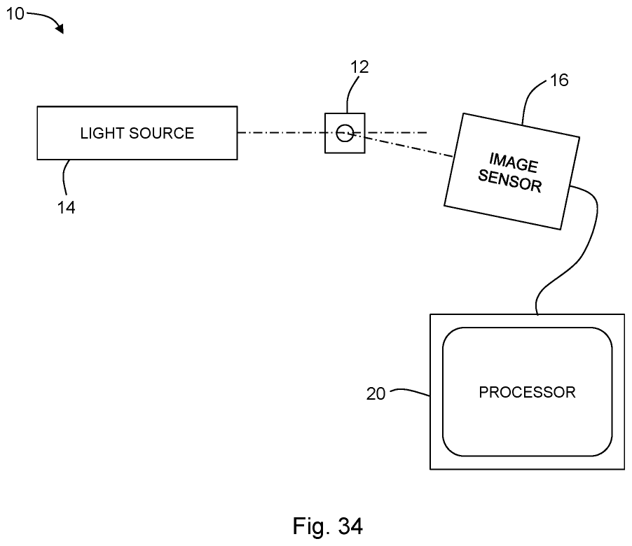

In another aspect, the present disclosure provides a system 10 for antibacterial susceptibility testing of a sample (see, e.g., FIG. 34). The system 10 includes a sample holder 12 and a coherent illumination source 14 configured to illuminate at least a portion of a sample within the sample holder 12. An image sensor 16 is positioned to receive light scattered by the sample thereby creating a series of speckle images over time. A processor 20 is in electronic communication with the image sensor 16. The processor 20 is configured to perform any of the methods disclosed herein. For example, the processor may be configured to receive from the image sensor the time series of speckle images; determine an inhibition status of the sample using a machine-learning classifier applied to the series of speckle images over time. The series of speckle images may be captured over any suitable measurement period, such as a measurement period of between 5-20 seconds, inclusive, (or shorter or longer measurement periods), such as, for example, a measurement period of 10 seconds.

In some embodiments, the processor is further configured to transform the series of speckle images over time into a frequency series of speckle images, and wherein determining the inhibition status of the sample uses the machine-learning classifier applied to the frequency series of speckle images. For example, in some embodiments, the processor is configured to determine an inhibition status of the test sample using the machine-learning classifier by classifying each pixel of the frequency series of images as either inhibited or not inhibited using the machine-learning classifier; calculating a percentage of pixels classified as inhibited; and determining an inhibition status of inhibited when the percentage of pixels classified as inhibited is greater than 50%, and an inhibition status of not-inhibited when the percentage of pixels classified as inhibited is less than or equal to 50%. In some embodiments, the frequency series of speckle images is normalized such that a DC term is normalized to 1. The machine-learning classifier may be an artificial neural network having a number of neurons in an input layer corresponding to a number of frequency components in the frequency series of speckle images.

In some embodiments, an optical axis of the image sensor is positioned in an angular range with respect to an optical axis of the illumination source defined by a Mie scattering model. The processor may be further configured to determine an average bacterium size of the sample using the Mie scattering model fitted with the series of speckle images.

In some embodiments, the processor is further configured to resize the series of speckle images. In some embodiments, the processor is further configured to determine an average intensity value of the series of speckle images.

The processor may be in communication with and/or include a memory. The memory can be, for example, a random-access memory (RAM) (e.g., a dynamic RAM, a static RAM), a flash memory, a removable memory, and/or so forth. In some instances, instructions associated with performing the operations described herein (e.g., operate an image sensor, capture a series of images) can be stored within the memory and/or a storage medium (which, in some embodiments, includes a database in which the instructions are stored) and the instructions are executed at the processor.

In some instances, the processor includes one or more modules and/or components. Each module/component executed by the processor can be any combination of hardware-based module/component (e.g., a field-programmable gate array (FPGA), an application specific integrated circuit (ASIC), a digital signal processor (DSP)), software-based module (e.g., a module of computer code stored in the memory and/or in the database, and/or executed at the processor), and/or a combination of hardware- and software-based modules. Each module/component executed by the processor is capable of performing one or more specific functions/operations as described herein. In some instances, the modules/components included and executed in the processor can be, for example, a process, application, virtual machine, and/or some other hardware or software module/component. The processor can be any suitable processor configured to run and/or execute those modules/components. The processor can be any suitable processing device configured to run and/or execute a set of instructions or code. For example, the processor can be a general purpose processor, a central processing unit (CPU), an accelerated processing unit (APU), a field-programmable gate array (FPGA), an application specific integrated circuit (ASIC), a digital signal processor (DSP), and/or the like.

In another aspect, the present disclosure may be embodied as a non-transitory computer-readable medium having stored thereon a computer program for instructing a computer to perform any of the methods described herein. For example, the non-transitory medium may have instructions for receiving from an image sensor a time series of speckle images; determining an inhibition status of the sample using a machine-learning classifier applied to the series of speckle images over time.

Some instances described herein relate to a computer storage product with a non-transitory computer-readable medium (which can also can be referred to as a non-transitory processor-readable medium) having instructions or computer code thereon for performing various computer-implemented operations. The computer-readable medium (or processor-readable medium) is non-transitory in the sense that it does not include transitory propagating signals per se (e.g., a propagating electromagnetic wave carrying information on a transmission medium such as space or a cable). The media and computer code (also can be referred to as code) may be those designed and constructed for the specific purpose or purposes. Examples of non-transitory computer-readable media include, but are not limited to: magnetic storage media such as hard disks, floppy disks, and magnetic tape; optical storage media such as Compact Disc/Digital Video Discs (CD/DVDs), Compact Disc-Read Only Memories (CD-ROMs), and holographic devices; magneto-optical storage media such as optical disks; carrier wave signal processing modules; and hardware devices that are specially configured to store and execute program code, such as Application-Specific Integrated Circuits (ASICs), Programmable Logic Devices (PLDs), Read-Only Memory (ROM) and Random-Access Memory (RAM) devices. Other instances described herein relate to a computer program product, which can include, for example, the instructions and/or computer code discussed herein.

Examples of computer code include, but are not limited to, micro-code or micro-instructions, machine instructions, such as produced by a compiler, code used to produce a web service, and files containing higher-level instructions that are executed by a computer using an interpreter. For example, instances may be implemented using Java, C++, .NET, or other programming languages (e.g., object-oriented programming languages) and development tools. Additional examples of computer code include, but are not limited to, control signals, encrypted code, and compressed code.

### EXAMPLES

The following examples provide descriptions of various non-limiting embodiments of the present disclosure.

### Materials and Methods

### Bacterial Culture:

Escherichia coli (E. coli) strain K-12 was used as a model bacterial strain in all experiments with ampicillin, gentamicin, and ceftriaxone. Multi-drug resistant (MDR) E. coli strain PS00278A (porcine isolate) was tested with ampicillin and gentamicin to contrast with the susceptible model bacterial strain. Staphylococcus aureus (S. aureus) strain PS00975A (human clinical isolate) was used with ampicillin to evaluate the methods' performance on a Gram-positive microorganism. Cultures were stored as a frozen stocks at −80° C. and resuscitated every 14 days to maintain a fresh inoculum. The culture was resuscitated by streaking onto Muller Hinton Agar (MHA) and was then incubated at 37° C. 20+/−2 h. A single colony from the MHA agar was re-streaked on a fresh MHA plate and incubated at 37° C. 20+/−2 h. A single colony from the MHA sub-streak plate was inoculated into 10 mL Muller Hinton Broth (MHB) and incubated at 37° C., shaking at 210 rpm, for 20+/−2 h. The overnight culture was diluted in MHB based on the OD₆₀₀ to obtain 5×10⁵ CFU/mL using a BioPhotometer D30 (Eppendorf, Hauppauge, N.Y.). An OD₆₀₀ of 1 was considered to be equal to 8×10⁸ CFU/mL, based on an E. coli OD₆₀₀-CFU/ml standard curve.

### Preparation of the Antibiotics:

Ampicillin (Sigma Aldrich, CAS# 7177-48-2) and gentamicin (Sigma Aldrich, CAS# 1405-41-0) stock solutions were prepared by dissolving antibiotic powder in sterilized MilliQ ultrapure water to achieve 5 mg/mL and 10 mg/mL stock solutions, respectively. All the stock solutions were frozen in 0.1 mL aliquots and stored at —20° C.

### Antimicrobial Susceptibility Testing Using Broth Microdilution:

The 5×10⁵ CFU/mL culture was used for broth microdilution and speckle imaging. For broth microdilution, the standard methods recommended by the Clinical and Laboratory Standards Institute (CLSI) guideline M100-S22 was applied to determine the MIC of ampicillin (AMP) and gentamicin (GEN) for E. coli strain K-12, MDR E. coli strain, and Staphylococcus aureus strain PS00975A. The CLSI guideline M100 ED30:2020 was used to define clinical resistance based on determined MICs. For both methods, 50 μL of MHB was aliquoted in wells of 96-well microtiter plates (Greiner bio-one). Antibiotics were added to the wells of the first row and sequentially diluted two-fold down the row. 50 μL of culture prepared as described above was added to each well and incubated for 16-20 hours. Negative and positive controls were included in each test plate and each test was carried out in three biological replicates per independent experiment and at least two independent experiments. MIC was determined by visually inspecting wells for turbidity resulting from culture growth.

### Verifying Antimicrobial Susceptibility Testing Using Time-Kill Kinetics:

Broth macrodilution was used to determine the time-kill kinetics of all strains, Culture in concentration of 5×10⁵ CFU/mL was first grown for 1 hour at 37° C. in a shaking condition (210 rpm) and was then exposed to antibiotic concentrations of 0×MIC (control), 1×MIC, and 2×MIC that were previously determined via broth microdilution. Viable cells were quantified at times 0, 1, 2, 3, 5, 8, 16, and 24 hours by spiral plating (easySpiral, Interscience) 10-fold dilutions onto MHB agar. Inoculated plates were incubated at 37° C. for 16-20 hours and counted to determine the CFU/mL at each time point. All experiments were completed using three biological replicates and three technical replicates per plate.

### Dynamic Laser Speckle Imaging (DLSI) Setup:

A helium-neon laser (wavelength: 632.8 nm, power: 0.8 mW, HNLS008L, Thorlabs Inc., USA) was used as an illumination source. The laser beam was slightly expanded by using a concave lens (focal length=−25.0 mm) before illuminating a cuvette (Fisherbrand, CAS#14-955-129) containing 3 mL of bacterial suspension. The resultant speckle pattern was captured by a CMOS camera (Zyla, ANDOR), interfaced to a computer. Note that other methods, such as an inverse telescope beam expander, can also be used to increase the incident laser beam size. Our choice of a diverging lens is due to its simplicity and compactness as well as the fact that an explicit physical model is not needed for the machine learning based analysis, which relaxes the requirement of a plane-wave incident beam as commonly used in dynamic light scattering. In addition, the diverging angle is small enough (˜1°), so that Mie scattering analysis (that assumes a planar incident wave and spherical scatterers) can still be used to estimate the scattering phase function in order to guide the optimization of the experimental setup, which, in this work, is configured to collect light scattering within an angular range of between 11° and 22°. This optimization is particularly important for increasing the sensitivity for measuring low-concentration samples which produce weak speckle patterns.

### Antibacterial Susceptibility Testing Using DLSI:

For DLSI-based AST, the culture grown as outlined above was adjusted to 5×10⁵ CFU/mL and was further incubated at 37° C., 210 rpm for 1 h to reach the logarithmic growth phase before an antibiotic was added to the culture in concentrations equal to 0×MIC (control), 0.5×MIC, 1×MIC, and 2×MIC. Three mL of each culture were collected at each time point, tᵢ (i=0, 1, . . . , 4) at 0, 30 min, 60 min, 90 min, and 120 min, respectively, and transferred into the cuvette used for DLSI in the imaging system described above. At each tᵢ, a dynamic speckle patterns series (16 bits, 1000×2000 pixels, 50 frames per second) was collected. The exposure time for each frame was 1 msec (millisecond) and the full capture period was 10 sec at each time point, tᵢ. All experiments were conducted in independent triplicates.

### Image Pre-Processing and Machine Learning Model:

The raw images (1000×2000) were first resized (100×200) using the nearest neighbor method to reduce the computational cost. The camera has a pixel size of 6.5 μm, corresponding to a 6.5 mm×13 mm detection area. Fourier Transform (FT) was performed along the time axis of the measured data cube. Only the spectral intensity was utilized in our analysis since a spectral intensity distribution with significant high frequency content generally corresponds to more active motion, whilst the spectral phase is not directly linked to motion activeness and presents a challenge in quantitative interpretation. This procedure transformed an original 100×200×500 data cube (two-dimensional space and one-dimensional time) into a new 100×200×249 data cube consisting of 249 positive frequency sampling points at each pixel position. The DC term was first normalized to 1 for each individual pixel spectrum and was subsequently removed. The negative frequencies provided identical information as their positive counterparts. Each measurement captured 20,000 spectra and each of the spectra contains 249 frequency features. This large data set was necessary for machine learning based analytics.

The experimental replicates are pooled, processed together, and then divided randomly into training, validation, and test groups. An Artificial Neural Network (ANN) with a hidden layer containing 300 neurons was constructed for classification prediction. The input layer contains 249 neurons, representing the frequency components, up to 25 Hz. The output was separated into 2 classes, “Inhibited” and “Non-Inhibited,” referring to either bacterial susceptibility or antibiotic resistance. The stochastic gradient descent (SGD) method was used to minimize the binary cross-entropy loss function. Binary classification for the “Inhibited” and “Non-Inhibited” bacteria groups was determined with the input from frequency domain for each pixel. All the image pre-processing and machine learning computations were performed with a desktop computer (Intel Core i9-9900k, 32GB RAM, NVIDIA RTX 2080 Ti). The training takes ˜2 minutes, and the FT analysis for one entire independent experiment takes ˜5 minutes.

### Results and Discussion

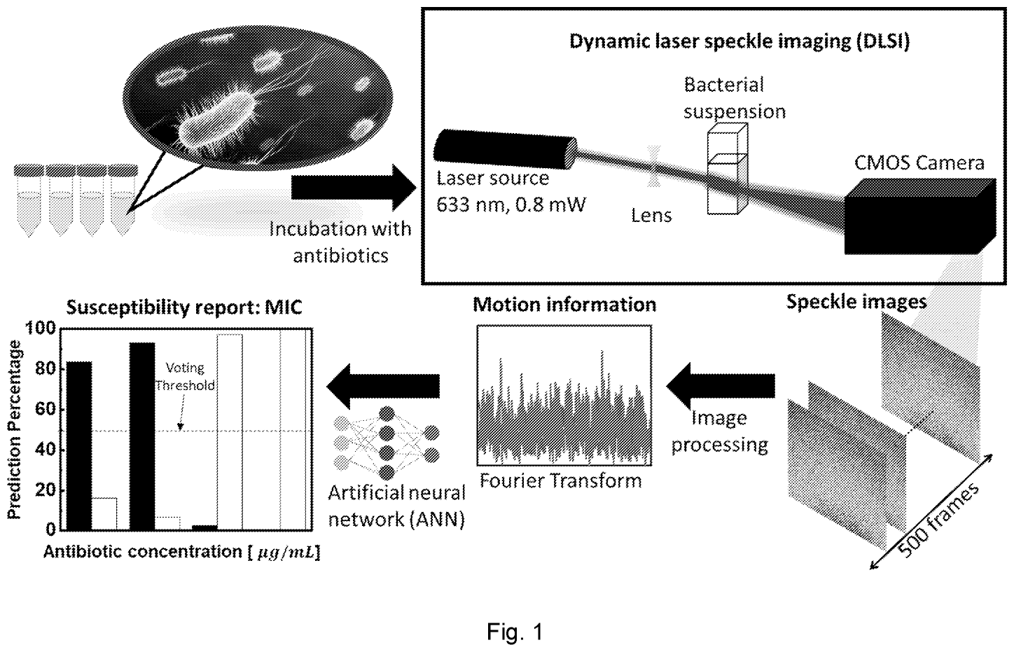

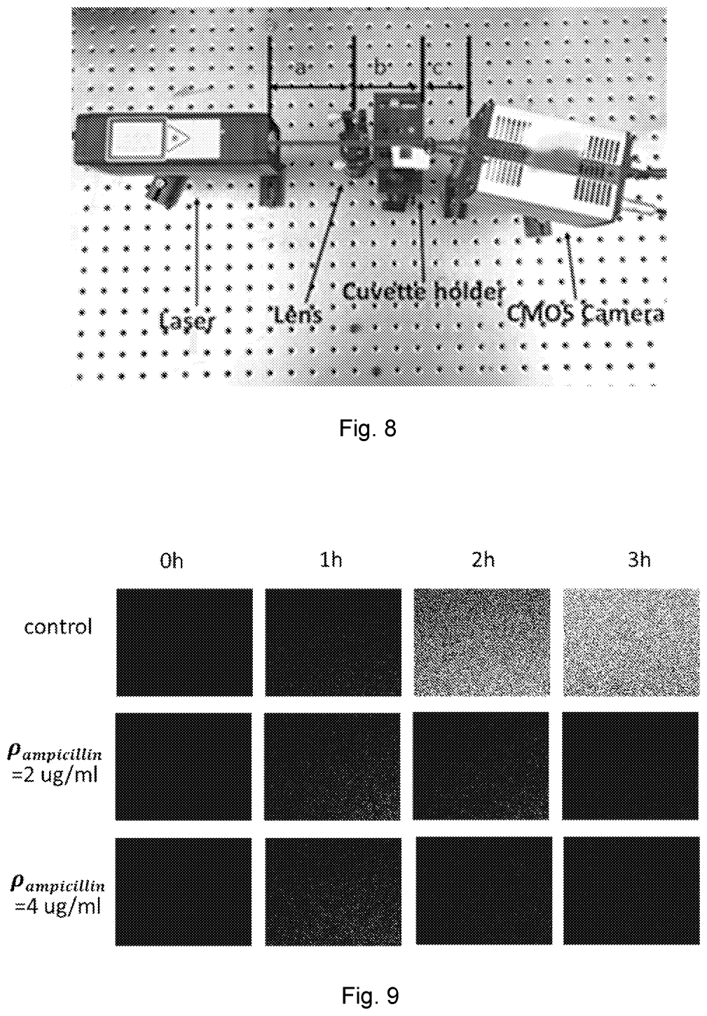

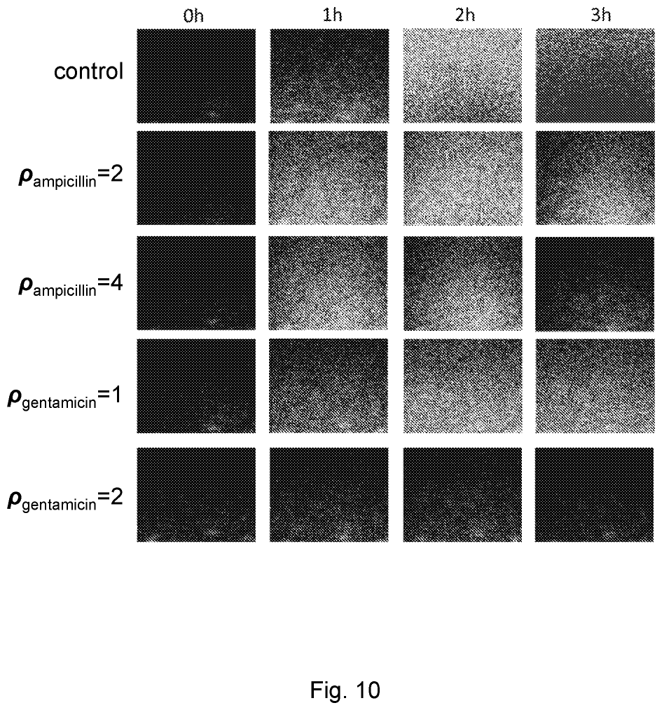

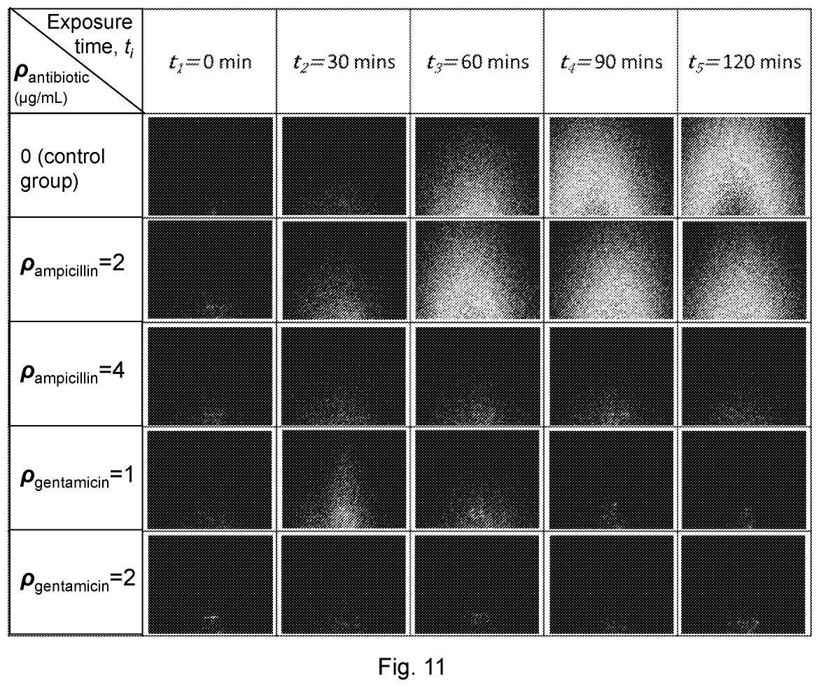

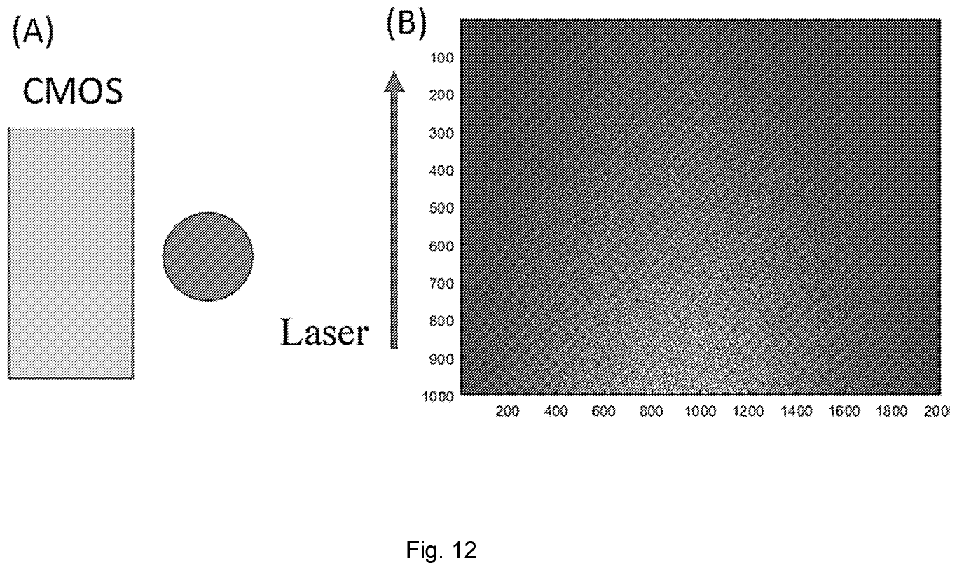

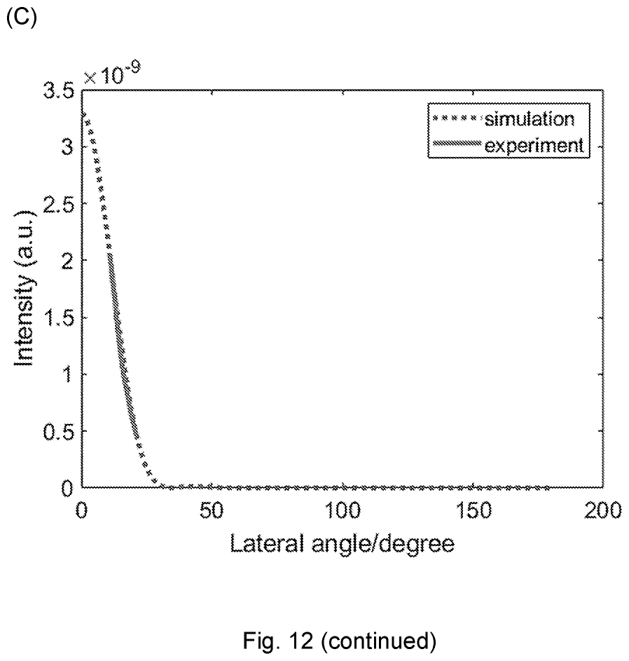

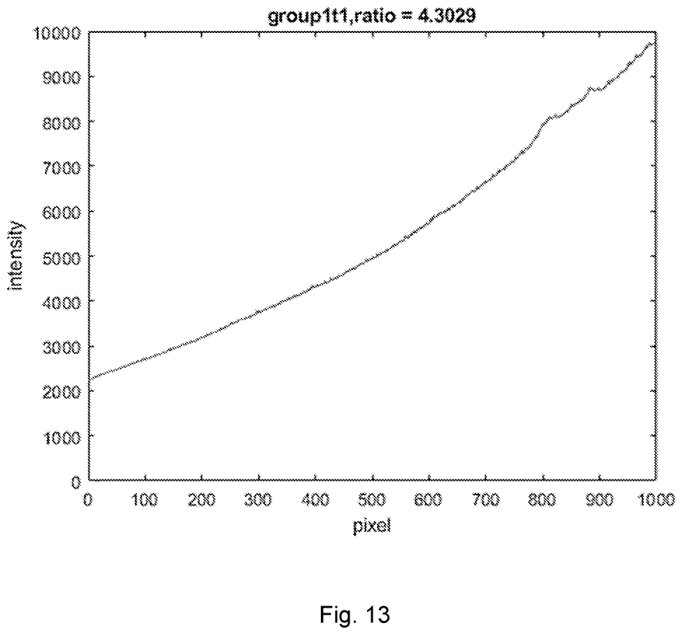

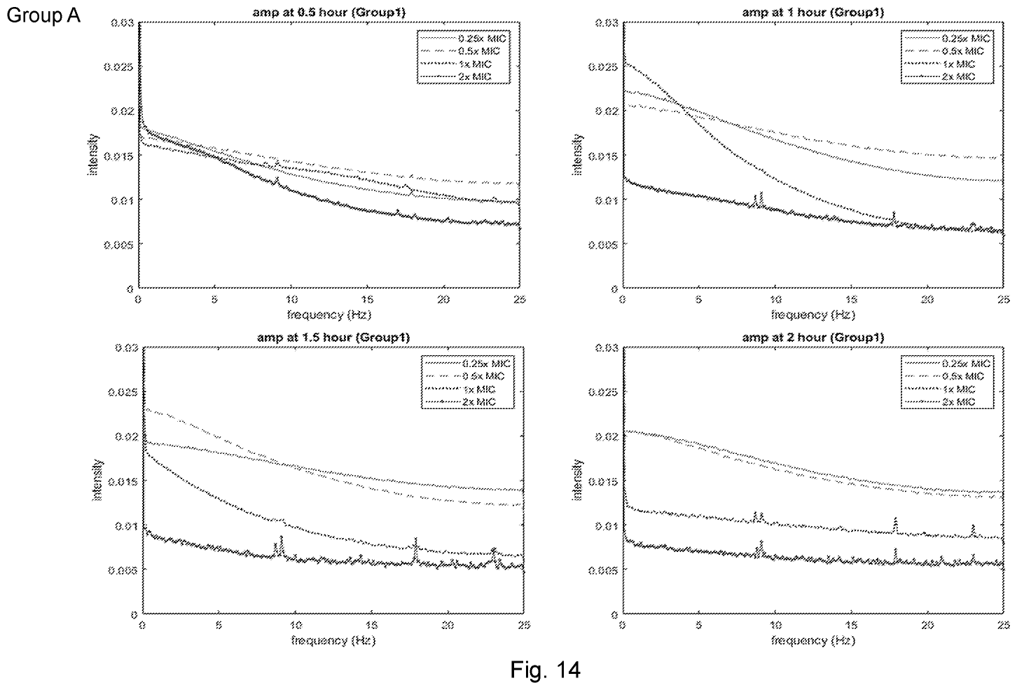

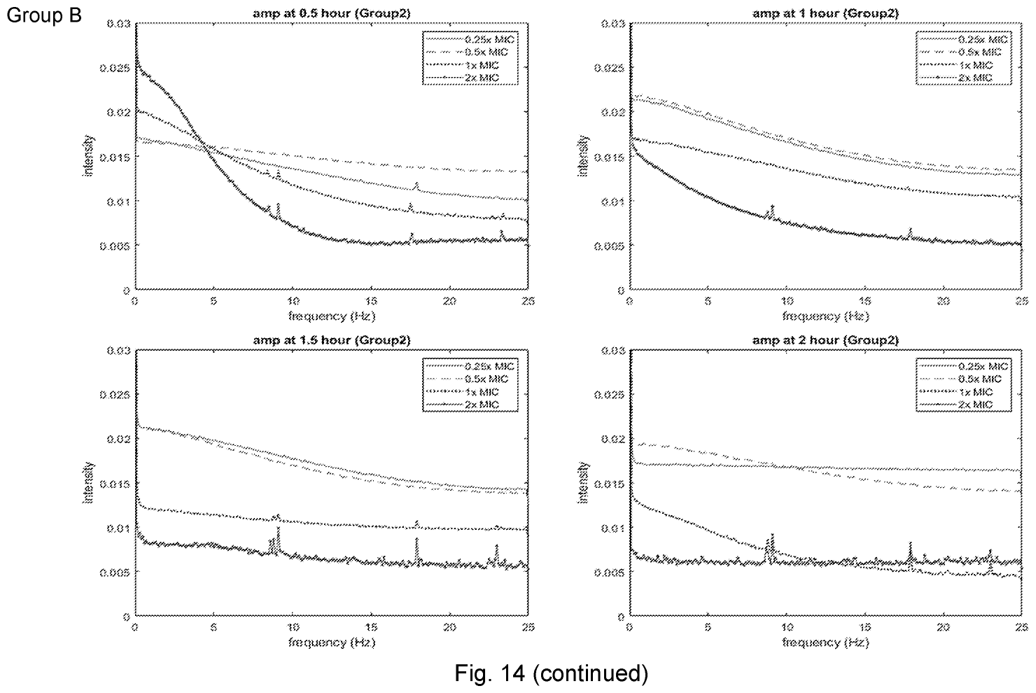

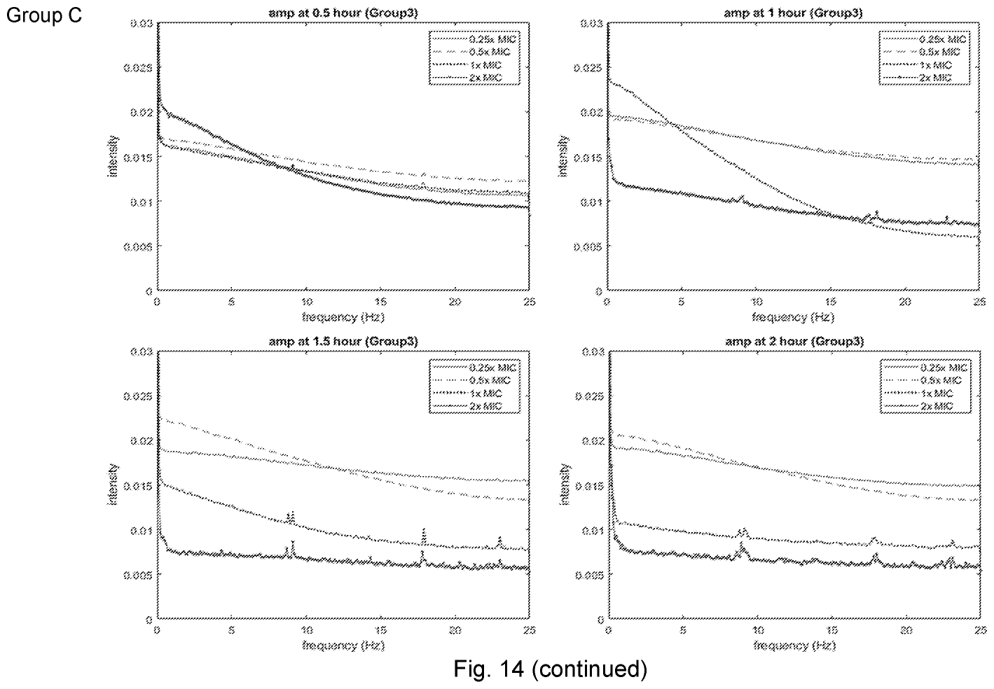

FIG. 1 shows a schematic of the DLSI setup consisting of a laser source, lens, cuvette holder, and camera. Distances between the setup components are listed in the Experimental Configuration section below (Table 1). The optical image of the setup is shown in FIG. 8. We have optimized the setup (in terms of the distance between different components and the scattered light angle, θ) guided by the Mie scattering model described below under the heading “Mie scattering analysis.” We investigated three configurations: Setting #1-3. An advantageous parameter in the setup is the angle between the axis of the camera and the laser beam (θ). The setup with Setting #3 (with θ˜15°) enables high sensitivity DLSI measurements, consistent with the Mie scattering theory (FIG. 12), while preventing the direct incidence of the laser beam on the camera which can saturate the image sensor. The results reported in the rest of the paper are based on this configuration, unless otherwise stated.

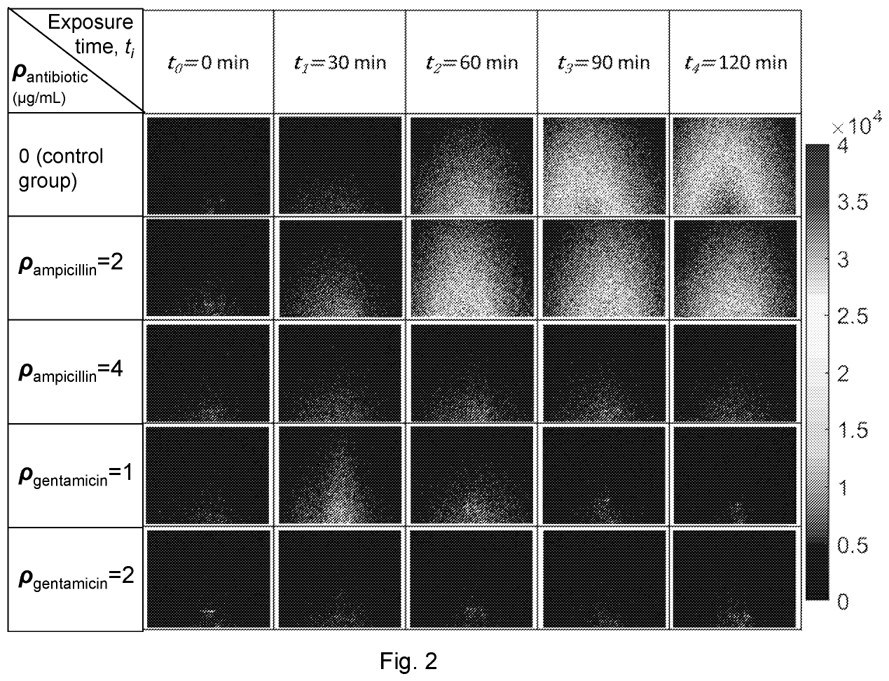

FIG. 2 shows typical speckle patterns of E. coli K-12 culture captured at tᵢ=0, 30, 60, 90, and 120 min with different concentrations of ampicillin and gentamicin (ρ_{AMP} and ρ_{GNT}), with respective MIC values of 4 μg/mL and 2 μg/mL (confirmed using gold standard methods; see Materials and Methods section). The speckle patterns for the control experiment (without antibiotics) are shown in the first row. In the absence of antibiotics, bacterial cells multiply. Their multiplication and division translate into an increase in the total number of scatterers, and hence, an increase of the intensity of the speckle patterns over time. At sub-inhibitory MIC concentration (e.g., 0.5×MIC value shown in the second and fourth rows, for AMP and GNT, respectively), there is an initial increase of the speckle intensity, followed by a slight decrease at 120 min. When cells are exposed to MIC (the third and fifth rows, for ampicillin and gentamicin, respectively), there is no significant change in the speckle intensity over time, which indicates inhibition of bacterial division.

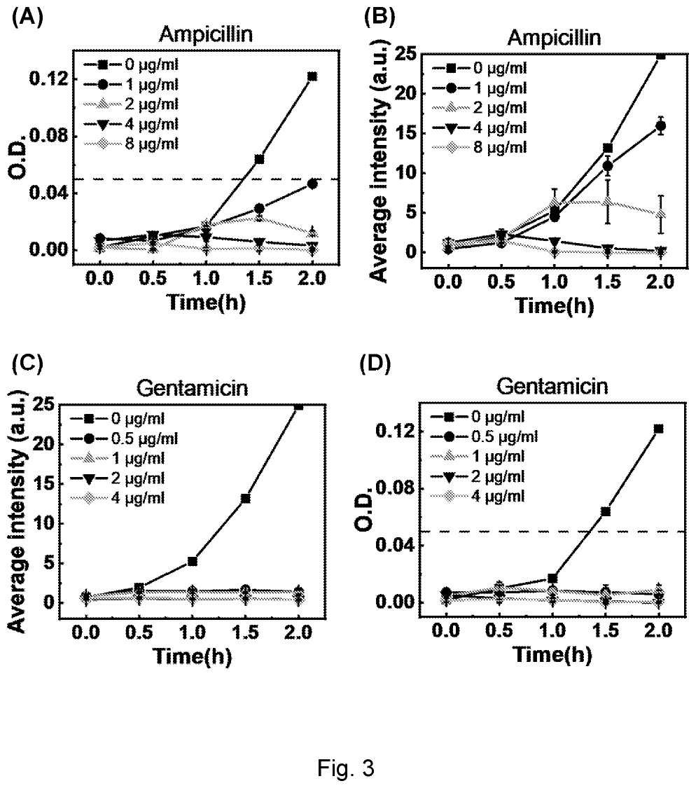

The OD data and the average intensity values obtained from raw speckle patterns (examples shown in FIG. 2) are shown in FIG. 3A-B and FIG. 3C-D for ampicillin and gentamicin, respectively. The average intensity for raw speckle image was measured on each pixelated image matrix collected at tᵢ's (hence, obtaining a data cube of 1000×2000 (pixels)×500 (# of frames)). It is worth mentioning that the average DLSI intensity captures time-kinetics of E. coli growth and is more sensitive than conventional OD measurements (with detection limit indicated by dashed lines in FIG. 3). While the OD values for different antibiotic concentrations are below the detection limit of the OD reading even after 120 min, the average speckle intensity values are distinguishable in 90 min.

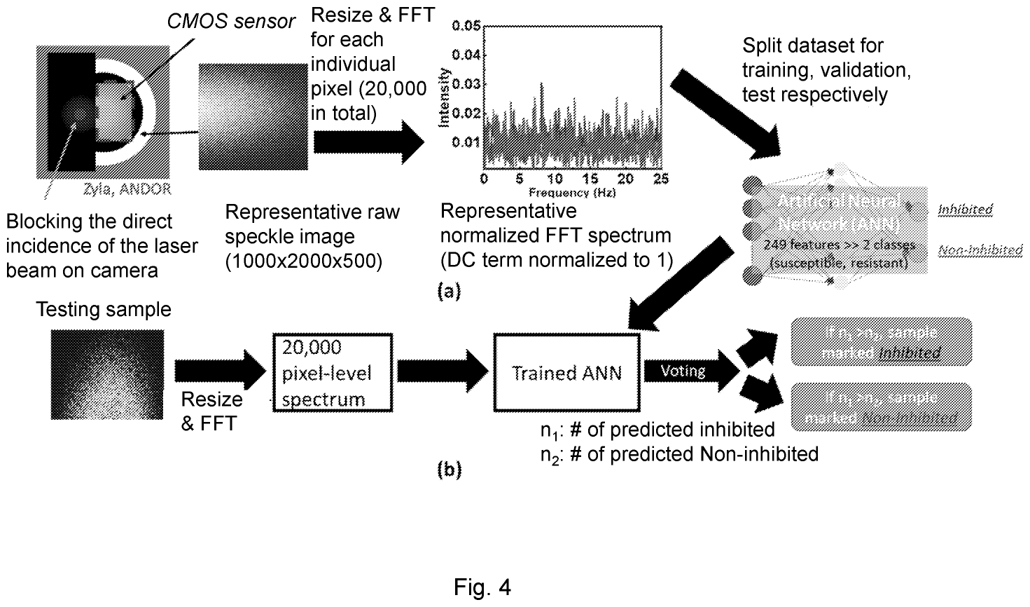

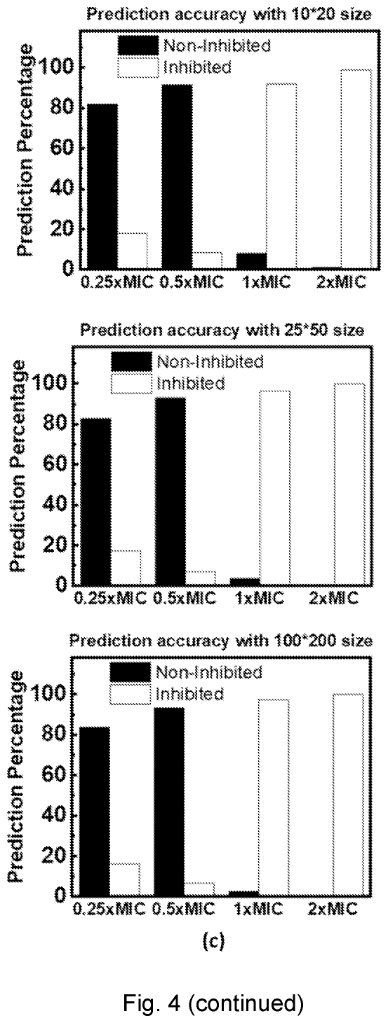

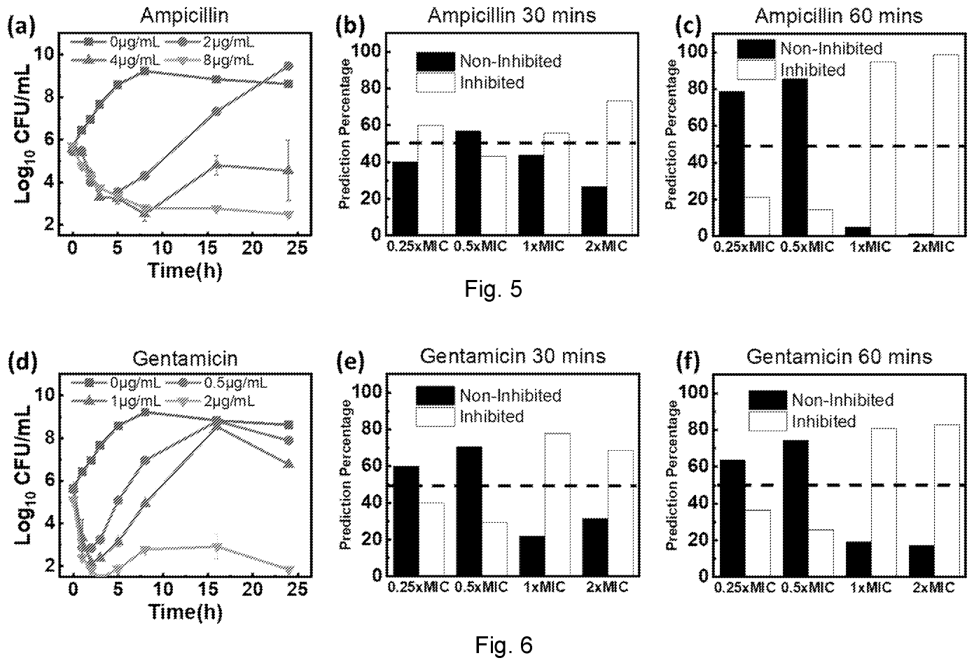

However, simple intensity measurements are “blind” to the overnight (>16 h) turbidity/colony count results (FIG. 3A/FIG. 5A and FIG. 3C/FIG. 6A for ampicillin and gentamicin, respectively), and prone to false positives; for example, intensity data determines ampicillin MIC as 2 μg/mL (FIG. 3), while the correct MIC based on the overnight data is 4 μg/mL (FIG. 5A). The intensity measurement also neglects the time-resolved motility information of cells. Moreover, absolute intensity is heavily influenced by the specifics of the optical setup (laser power, exposure time, camera gain, etc.) This presents a challenge to calibrate and extend the methodology to a generalized case. To overcome this problem, we utilize machine learning and train artificial neural network (ANN) models using the gold-standard method overnight results to analyze the dynamic speckle patterns and determine MIC. FIG. 4 illustrates our methodology for preprocessing the data and applying the machine learning algorithm. As discussed earlier, the experimental setup was optimized using the Mie scattering theory to capture dynamic speckle patterns over an angular range between 11° and 22° (with Setting #3 set at θ˜15°). The transmitted laser beam was blocked to avoid saturation of the camera and ensure collection of high-quality speckle patterns. As shown in FIG. 4A, Fourier Transform analysis was performed along the time axis on individual pixels, with the DC component (direct current component which is the value at 0 Hz) normalized to 1 (details provided below under the heading “Fourier Transform (FT) analysis”) to ensure that the analysis is independent of the absolute speckle intensity values. For each time point, tᵢ, of each group, we obtained 20,000 pixel-level spectra, each consisting of 249 frequency features. We then built a separate ANN model for each time point, with 249 neurons at the input, 300 hidden units, and 2 output neurons for binary classification.

We trained the ANN models with experimental data for E. coli K-12 exposed to antibiotics of different concentrations (0.25×, 0.5×, 1×, and 2×MIC). We conducted experiments in 3 independent replicates with groups exposed to four concentrations for each antibiotic. The four concentrations yielded a total of 80,000 spectra for each time point. As shown schematically in FIG. 4B, we split the samples randomly into training, validation, and test groups with ratios of 70%, 15%, and 15%, respectively. The validation was performed to avoid model overfitting and the test group was used to verify the predictive power of the trained neural networks. Two independent experiments, with each experiment utilizing four different concentrations of the antibiotic which result in a combined data size of 160,000 samples at each time point, were used in building the learning models. We then used a third, independent experiment that consisted of 80,000 spectra at each time point for a final comprehensive test to evaluate the robustness and accuracy of the trained models.

The overnight OD values were utilized to label the dataset. If the OD after overnight incubation was at the same level as the initial OD, the bacterial group was labeled as “Inhibited”. On the other hand, if the final OD was higher than the initial OD value (above the resolution limit of the equipment which is ˜0.05), the group was labeled as “Non-Inhibited”. In our experiments, 0.25× and 0.5×MIC were labeled as “Non-Inhibited”, while 1× and 2×MIC were labeled as “Inhibited”. For each group at time point tᵢ, the result of the test is interpreted using a voting process where the percentage of each prediction category indicates the likelihood for this group to be classified as the said category. For example, 30% “Inhibited” implies 30% of the 20,000 spectra are predicted “Inhibited,” while the remaining 70% are predicted to be “Non-Inhibited.” Therefore, this group would be classified as “Non-Inhibited”. As shown in FIG. 4C, we also evaluated the effect of the resizing factor on the performance (prediction percentage) of the machine learning analysis. The black bar indicates the percentage of pixels “Non-Inhibited,” while the red bar indicates the percentage of “Inhibited.” With 100×200, 25×50, and 10×20 resizing factors, we observed only a 5% difference among the machine learning results. Furthermore, they all correctly predict MIC in 60 min through the voting strategy using a 50% threshold for decision-making. The ability to reduce the data size using a higher resizing factor indicates the required computational resources can be reduced without significant performance degradation. It should be noted that the ML method does not rely on the absolute speckle intensity which could be difficult to calibrate. Detailed discussion on machine learning and confusion matrices (which allow visualization of the ANN performance) are provided below under the heading “Confusion matrix for the ANN model and time-evolution.”

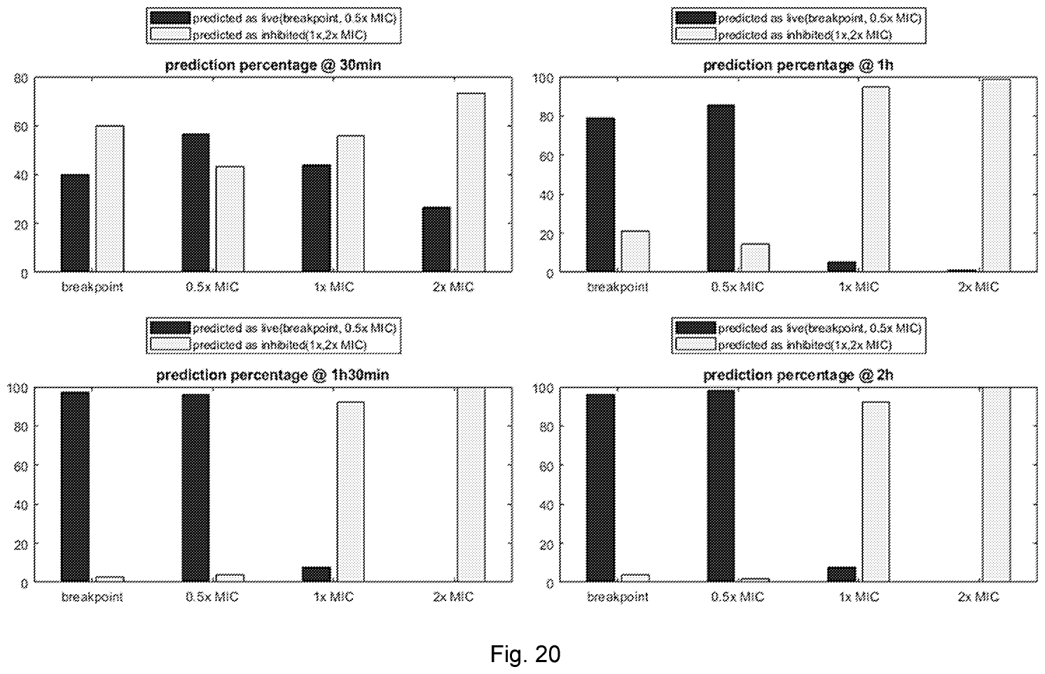

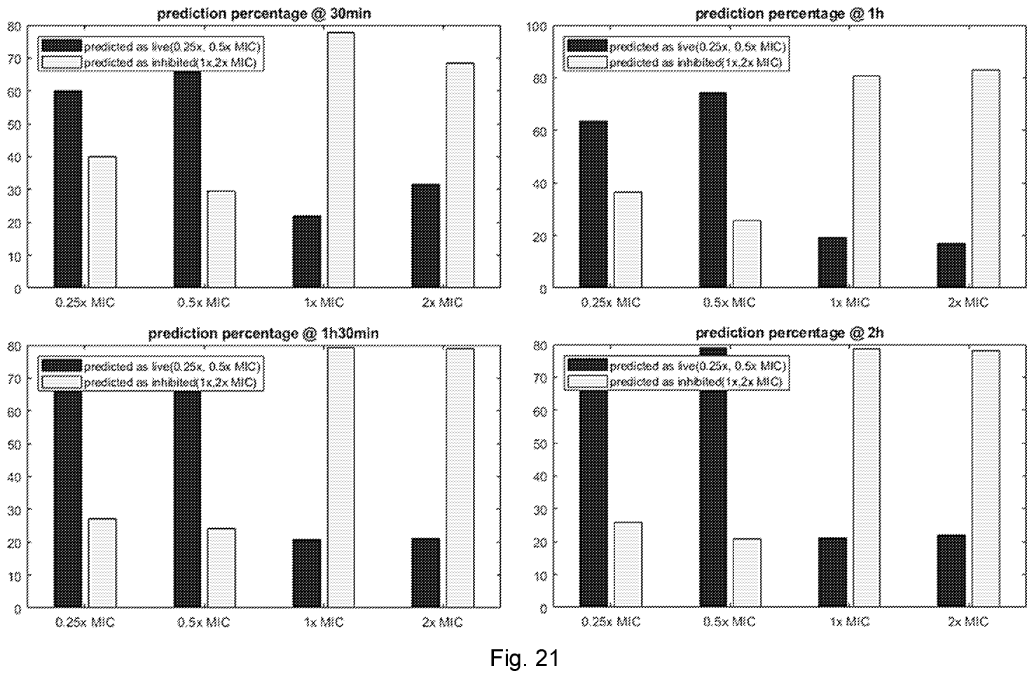

The machine learning results for the prediction of ampicillin and gentamicin susceptibility and their MIC values are shown in FIGS. 5 and 6, respectively. FIG. 5A and FIG. 6B show the time-kill curves obtained using the broth macrodilution method. The MIC was defined as the minimal concentration at which bacterial population remained at or below the level of initial inoculum concentration. Hence, if at a given antibiotic concentration the culturable bacterial population substantially decreased in initial hours, but later on resumed growth and surpassed the initial inoculum concentration, that antibiotic concentration was considered sub-inhibitory. The pixel-level prediction percentage obtained using machine learning is shown in FIG. 5B-C and FIG. 6B-C, for ampicillin and gentamicin, respectively. FIG. 5B suggests that 30 min is not long enough for accurate prediction of MIC. We defined the final classification result based on the voting strategy described above: the dashed lines in FIGS. 5 and 6 indicate the voting threshold to predict antimicrobial susceptibility. A correct prediction means classification of “Inhibited” for 1×MIC and 2×MIC, while at the same time, 0.25×MIC and 0.5×MIC should be classified as “Non-Inhibited”. For example, at 30 min, the predicted percentage of classification as “Inhibited” for 2×MIC of AMP is ˜50%, making it difficult to decide in which class/label the sample belongs to. While at 60 min, the correct classification (“Inhibited”) is predicted in more than 80% of the votes. Interestingly, by comparing FIGS. 5 and 6, it is observed that the method can predict MIC for gentamicin in 30 min (compared to 60 min for AMP). Hence, for accurate analysis of DLSI data and identifying MIC, DyRAST required a minimum of 60 min in our studies with ampicillin and gentamicin. The pixel-level prediction percentage difference increased with time, as shown in FIG. 20 and FIG. 21 for ampicillin and gentamicin, respectively.

It is worth noting that by counting viable cells using the broth macrodilution technique (a sensitive yet slow and laborious method), differentiation among 0.5×MIC, 1×MIC and 2×MIC was possible only after 5 hours for ampicillin and after 2 hours for gentamicin (see FIGS. 5A and 6A) in our studies. The only alternative rapid phenotype-based method available on the market and approved by the FDA, requires at least a 6-hour culture incubation to confidently determine the antimicrobial susceptibility. It should be noted that the difference between speckle imaging results and the time-kill results (initially seeming inhibitory for sub-MIC concentrations) could be due to viable but non-culturable cells (VBNC). VBNCs are live and active when the speckle images are captured but cannot survive the plating process, as the plating method quantifies the time after the cells transferred to a plate. Yet, DyRAST can accurately determine a MIC of ampicillin and gentamicin for E. coli K-12 within 1 hour of culturing an isolate (despite the initial seemingly inhibitory effect of sub-MIC concentration). This is a distinct feature of our system to determine the MIC correctly despite the potential gradual adaptation of bacteria to antibiotics, which may lead to a false diagnosis if overlooked.

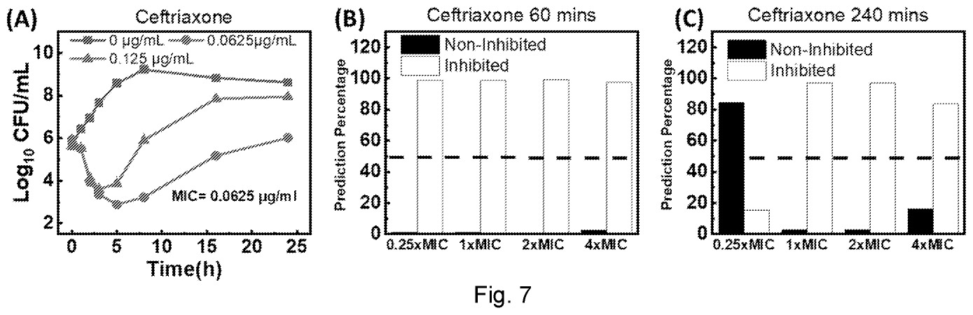

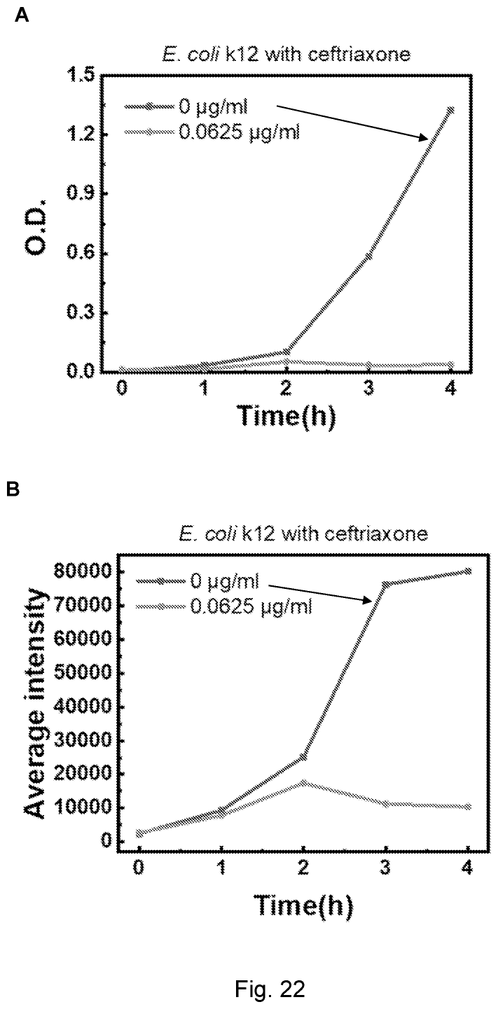

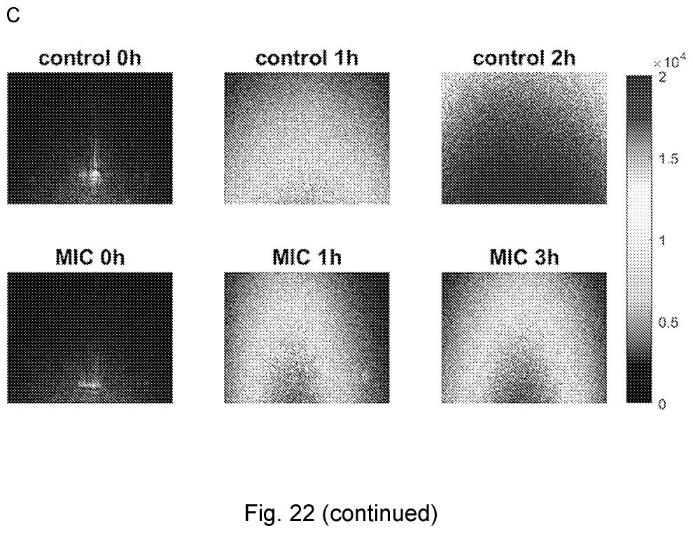

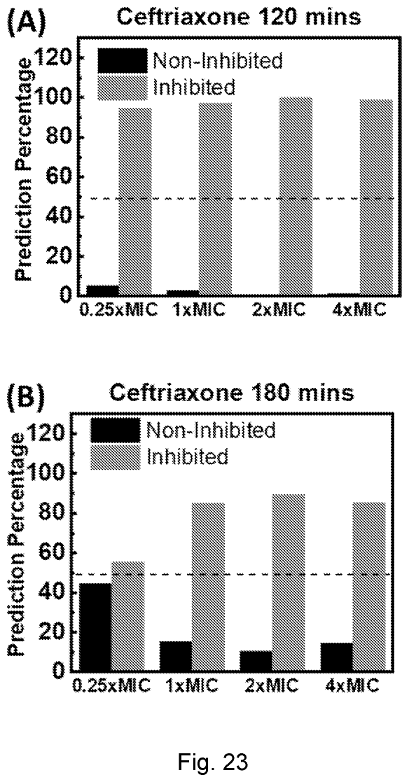

We also studied the performance of DyRAST for determining MIC of ceftriaxone (a clinical antibiotic) on E. coli K-12. OD₆₀₀ curves, raw speckle images, and average speckle intensity values for control cultures and cultures treated with MIC (determined using BMD method) are provided in FIG. 22. FIG. 7A plots the time-kill curves for control, MIC, and 2×MIC of ceftriaxone. FIG. 7B-C depict the prediction percentage results at 60 min and 4hours obtained using the ANN model, suggesting that at least 4 hours is needed to determine ×MIC of ceftriaxone. The longer assay time can be associated to ceftriaxone characteristic, a third-generation cephalosporin with longer lag time, which means a longer time is needed to affect the bacteria. FIG. 23 summarizes the ANN prediction percentage results for other time points, indicating that accuracy increases with time, similar to the previous cases with ampicillin and gentamicin.

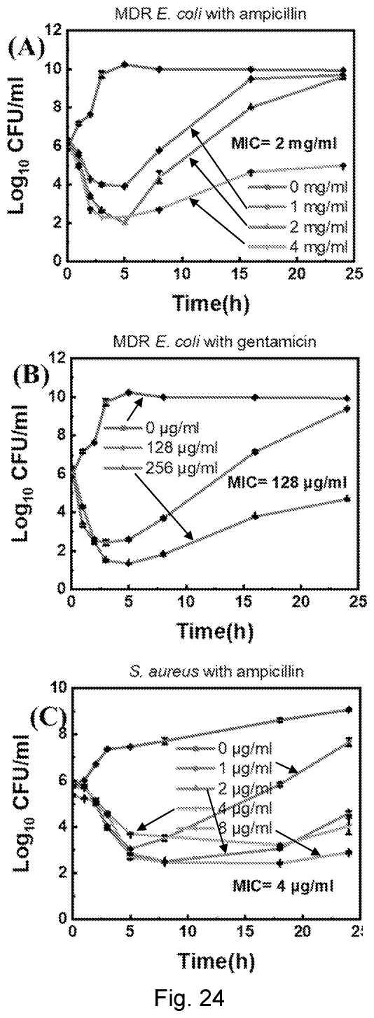

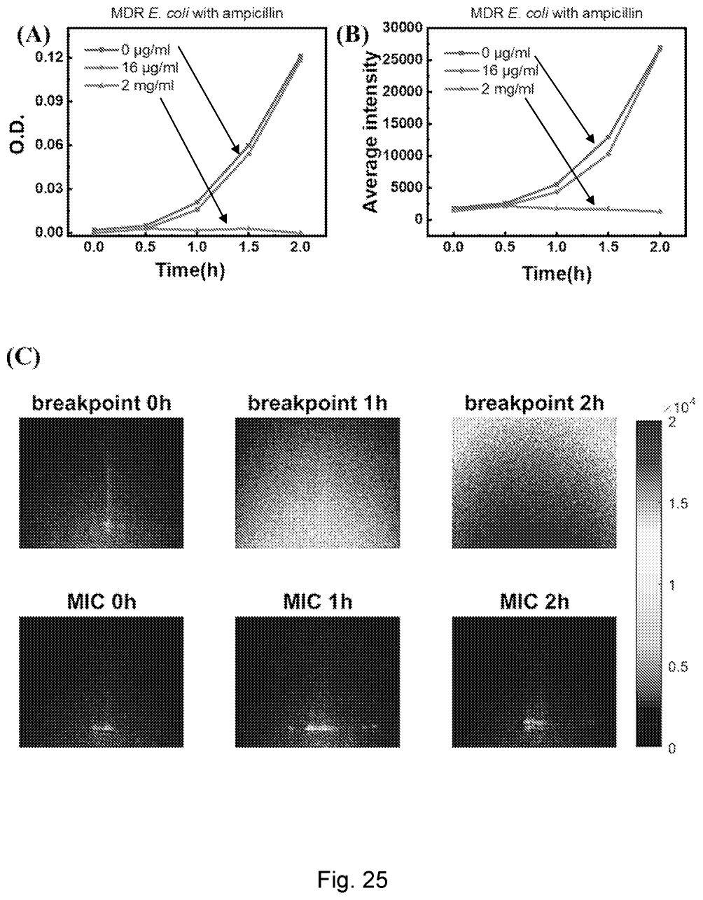

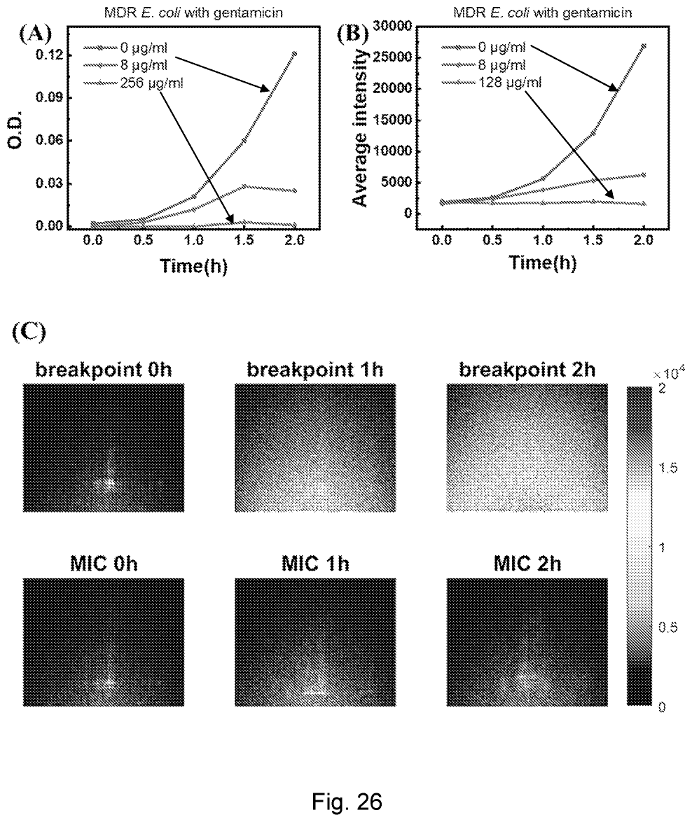

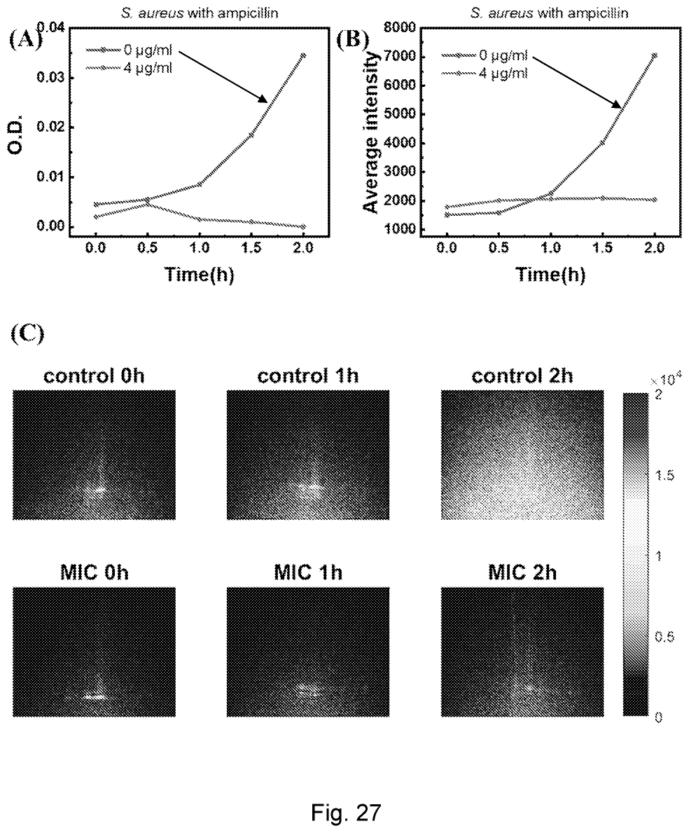

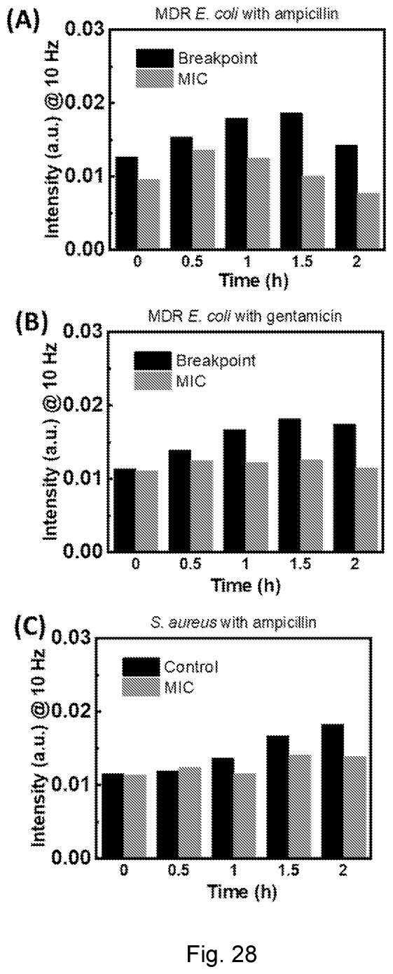

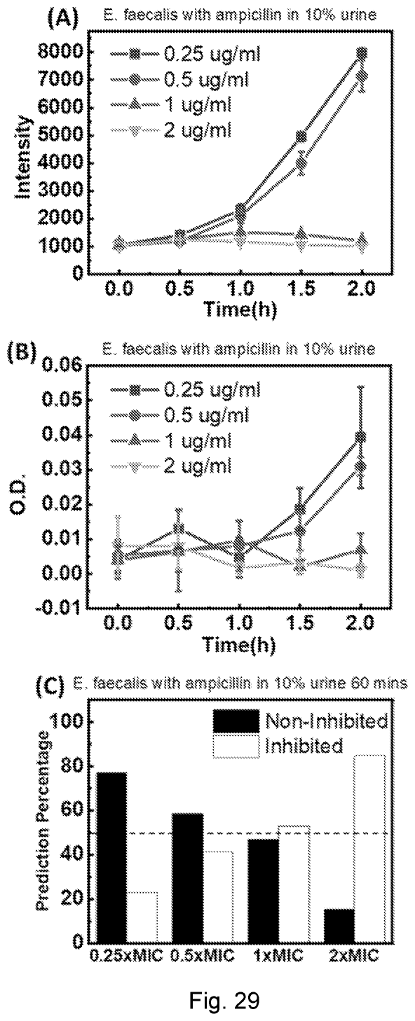

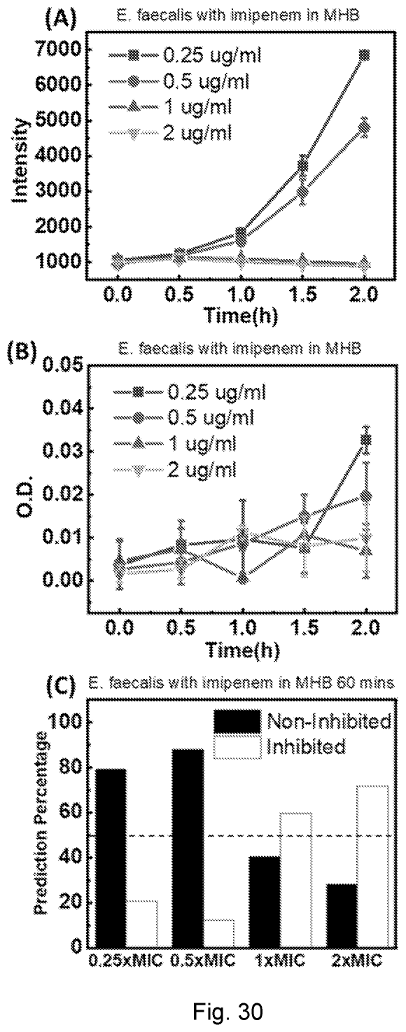

In order to further demonstrate applicability of the DLSI system for monitoring bacterial viability beyond E. coli K-12, we performed preliminary experiments with a MDR E. coli (treated with ampicillin and gentamicin) and a clinical isolate of S. aureus treated with ampicillin. Time-kill curves for these strains are plotted in FIG. 24. OD₆₀₀ curves, raw speckle images, and average speckle intensity values are provided in FIG. 25 (for MDR E. coli treated with ampicillin), FIG. 26 (for MDR E. coli treated with gentamicin), and FIG. 27 (for S. aureus treated with ampicillin). For MDR E. coli, we also tested a concentration of ampicillin and gentamicin that serves as a clinical resistance diagnosis breakpoint (intermediate breakpoint concentration: 16 μg/mL for ampicillin and 8 μg/mL for gentamicin) determined by the CLSI guideline (M100 ED30:2020). FIG. 28A-C depict the time-evolution of the Fourier component at 10 Hz for MDR E. coli treated with ampicillin and gentamicin (breakpoint concentration and MIC) and S. aureus treated with ampicillin (control and MIC), respectively. A larger FT component indicates more dynamic scattering, and hence a higher rate of change (e.g., due to bacterial movement). These results suggest that laser speckle imaging, when combined with machine learning, is able to differentiate between viability under inhibitory and non-inhibitory conditions (in 1 hour for S. aureus and MDR E. coli and in 4 hours for ceftriaxone). For MDR E. coli, our method enabled identifying the resistance against ampicillin and gentamicin in one hour (based on the data for the breakpoint concentrations).

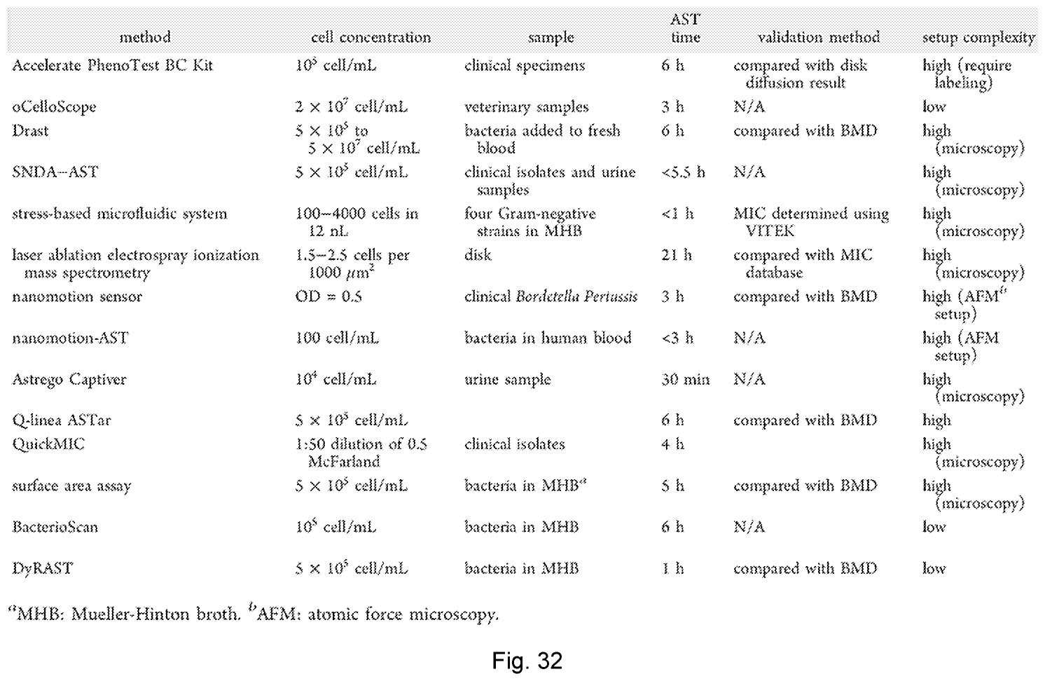

FIG. 32 compares the performance of DyRAST reported in this study with other size/motion RASTs based on the initial cell concentration, RAST time, sample condition, and setup complexity. In contrast with methods based on microfluidics or other methods (e.g., based on atomic force microcopy), DyRAST does not require expensive instrumentation and utilizes similar tools as the existing AST assays which are already formulated, verified, and validated and are widely available at a relatively low cost. Therefore, the proof-of-concept method presented here may offer a promising route for developing a portable, affordable, and automated RAST for application in resource-limited areas, which is critical in our global fight for the AMR stewardship. Toward this goal and to translate the proof-of-principle studies in this work to clinical settings, a detailed analysis using a library of various clinical strains and different antibiotics is required. Moreover, future extension of this work include study of more complex media (such as urine) and optimization of the DyRAST system to enable analysis of lower initial bacterial concentration (starting from 1000 CFU/mL which is relevant for urinary tract infections) without sacrificing the assay time.

### Further Results

In addition to demonstrating performance of DyRAST with E. coli (a Gram-negative bacterium) in culture medium (MHB), we demonstrated the method for rapid AST on Enterococcus faecalis (E. faecalis; a Gram-positive bacterium). Since E. faecalis is usually resistant to aminoglycosides (e.g., gentamicin), we used the β-lactam antibiotic, ampicillin and imipenem, to characterize DyRAST's performance.

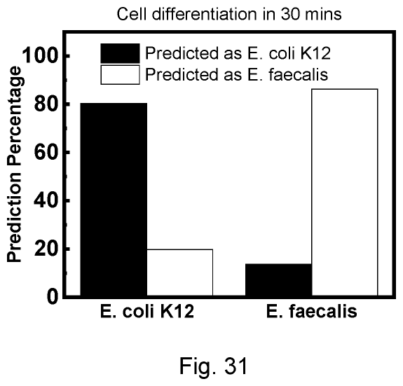

In addition, to demonstrate application of the method for direct AST of urine samples, we performed analysis in spiked urine (10% human urine sample diluted in MHB). The intensity analyses, optical density (OD) values, and machine learning (ML) results of dynamic laser speckle imaging (DLSI) data with E. faecalis treated with ampicillin in 10% urine and imipenem in MHB are shown in FIGS. 31 and 32, respectively. These results confirm that ML-powered DyRAST can predict MIC in 60 min in spiked urine samples without any sample preparation.

We also applied the same machine learning model for analysis of DLSI data for differentiating between E. coli and E. faecalis. Our results in FIG. 33 show that the method is able to distinguish between the two pathogens (with different Gram stains and cell shape) in 30 minutes.

The preparation of the ampicillin stock solution has been discussed in the published article (ACS Sens. 2020, 5, 10, 3140-3149). The Enterococcus faecalis (ATCC 29212) was purchased from Fisher Scientific (KWIK-STIK). The overnight culture of E. faecalis in the Muller Hinton Broth (MHB) as discussed in the published article. 0.5 mg/mL imipenem (Sigma Aldrich, CAS# 74431-23-5) stock solution was prepared by dissolving the antibiotic powder in sterilized MilliQ ultrapure water. The stock solution was prepared every time before the tests due to instability of the aqueous solution. The 10% urine medium was prepared by mixing the single donor human urine (from Innovative Research) with MHB (1:9 V/V). The MIC determination, dynamic speckle imaging setup, and the data processing method are the same as the previous report.

It will be recognized from the foregoing that this disclosure provides a rapid, phenotype-based antibacterial susceptibility testing method capable of identifying MIC in 60 minutes. The method leverages machine learning analysis of time-resolved dynamic laser spackle imaging (DLSI) patterns to predict antimicrobial susceptibility and MIC in a rapid and reliable manner. The DLSI data was collected using a simple-to-use, low-cost optical setup, with no labeling or advanced imaging/optical setup required. To demonstrate the capabilities of the method (termed as DyRAST), we studied the effect of two antibiotics, ampicillin and gentamicin, which have different mechanisms of action. DLSI captures change of bacterial motion/division in response to antibiotic treatment. The optical setup was optimized by using the Mie scattering theory to extract maximum information from collected data in a rapid manner. The DyRAST was validated against the gold standard AST methods using E. coli K-12 as a model microorganism. By adapting a voting strategy for analysis of the prediction values obtained using the artificial neural network model, the method predicted MIC with 100% accuracy in all tested conditions. The technique can be optimized for analysis of other (pathogenic) bacterial and fungal species and their response to antimicrobial treatment. Moreover, considering no complicated optical components are required for speckle imaging, a portable version of DyRAST can be potentially adapted by using consumer-level components, such as smartphone camera and laser diodes.

### Experimental Configuration

TABLE 1
The optical modules position parameters.

| Setting | a | b | c | θ |
|---|---|---|---|---|
| #1 | 9 cm | 6 cm | 8 cm | 20° |
| #2 | 9 cm | 6 cm | 4 cm | 10° |
| #3 | 9 cm | 5 cm | 6 cm | 15° |

a: distance between the lasers and lens,
b: distance between the lens and cuvette,
c: distance between the cuvette and camera.
θ: the angle between the camera optical axis and the laser beam.

### Mie Scattering Analysis

In the Mie scattering simulations, we calculate the scattering light at far field from the incident laser light. S terms are the scattering elements. In our model, we consider the incident laser wavelength, particle size, the refractive index of the particle, and the refractive index of the medium.

$$[ \begin{matrix}{E}_{\vert \vert S} \\ {E}_{⊥ S}\end{matrix} ] = \frac{{e}^{- i k ( r - z )}}{- i k r} [ \begin{matrix}{S}_{2} & {S}_{3} \\ {S}_{4} & {S}_{1}\end{matrix} ] [ \begin{matrix}{E}_{\vert \vert i} \\ {E}_{⊥ i}\end{matrix} ]$$

Next, we used the model to estimate the particle size based on the experimental data collected using DLSI.

According to Table 3, the larger the fitted ratio, the larger the estimated particle size. Based on to Table 2, we can observe that for AMP below MIC, where the bacterium is not inhibited, the fitted size is higher. In this fitting, we model the particle as a spherical object. We can infer the average size information from the captured speckle imaging based on the Mie scattering model. So, this indicates that the effective size of the initial bacteria is around 0.55 μm, for non-inhibited cells is 0.57 μm, and for inhibited cells is 0.44 μm in radius. While for GEN, there is no significant change for the measured ratio because of its different mechanism compared to AMP.

TABLE 2
Calculated ratio of the 1,000^{th} pixel to the 1^{st} pixel for different antibiotic
concentrations at different time points. We use the ratio to fit the particle size
based on Mie scattering model.

| | | time | | | | |
|---|---|---|---|---|---|---|
| ratio | | t0 | t1 | t2 | t3 | t4 |
| Group | 0.25 × MIC | 3.131207 | 3.830889 | 4.302889 | 4.331992 | 4.495746 |
| (AMP) | 0.5 × MIC | 3.221468 | 3.849928 | 4.191163 | 4.555702 | 4.465895 |
| | 1 × MIC | 2.737577 | 3.547474 | 3.819612 | 3.200287 | 2.855632 |
| | 2 × MIC | 2.406889 | 3.417774 | 2.760083 | 2.442261 | 2.200918 |
| | | time | | | | |
| ratio | | t0 | t1 | t2 | t3 | t4 |
| Group | 0.25 × MIC | 2.606152819 | 3.586024394 | 3.61690019 | 3.844444251 | 3.578184093 |
| (GEN) | 0.5 × MIC | 3.072343756 | 3.474386188 | 3.615405652 | 3.415825336 | 3.385749959 |
| | 1 × MIC | 2.9127119 | 3.02019634 | 2.766714056 | 3.081785913 | 3.058094747 |
| | 2 × MIC | 2.819867412 | 2.953992592 | 2.958340363 | 2.809487365 | 2.682107782 |

TABLE 3
The fitted size with respect to the ratio

| Ratio | Fitted radius (m) |
|---|---|
| 2.3-2.4 | 0.44 × 10⁻⁶ |
| 3.75-3.85 | 0.55 × 10⁻⁶ |
| 4.3-4.5 | 0.57 × 10⁻⁶ |

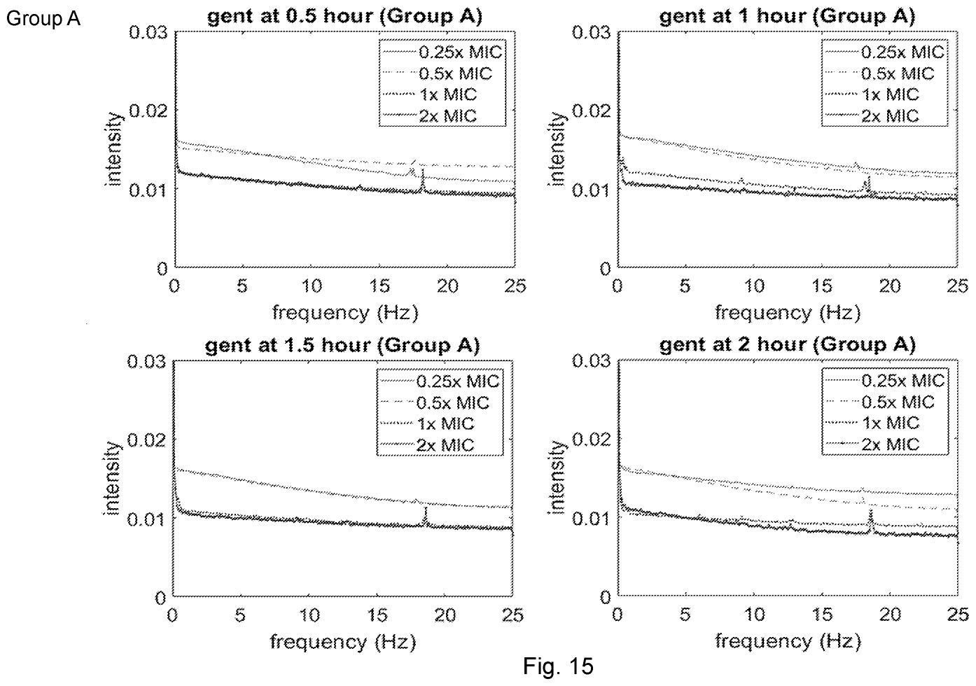

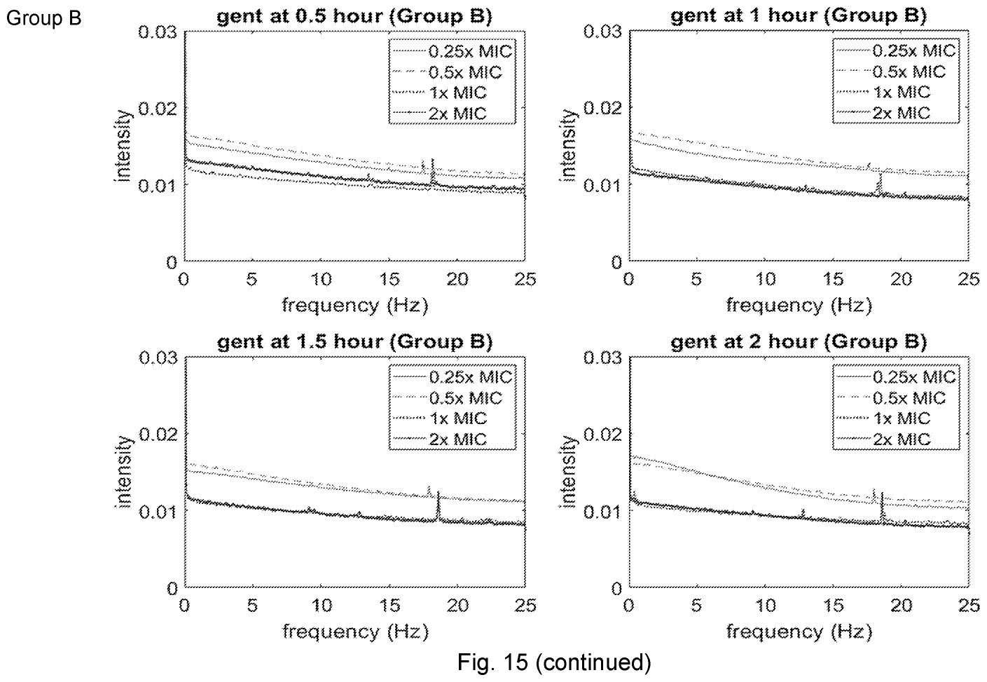

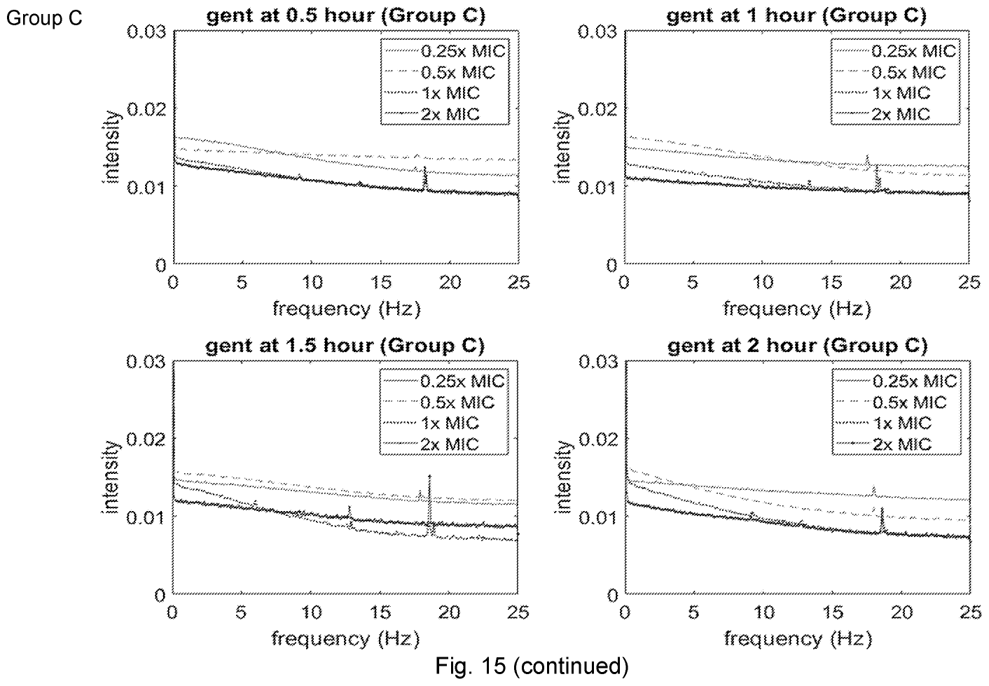

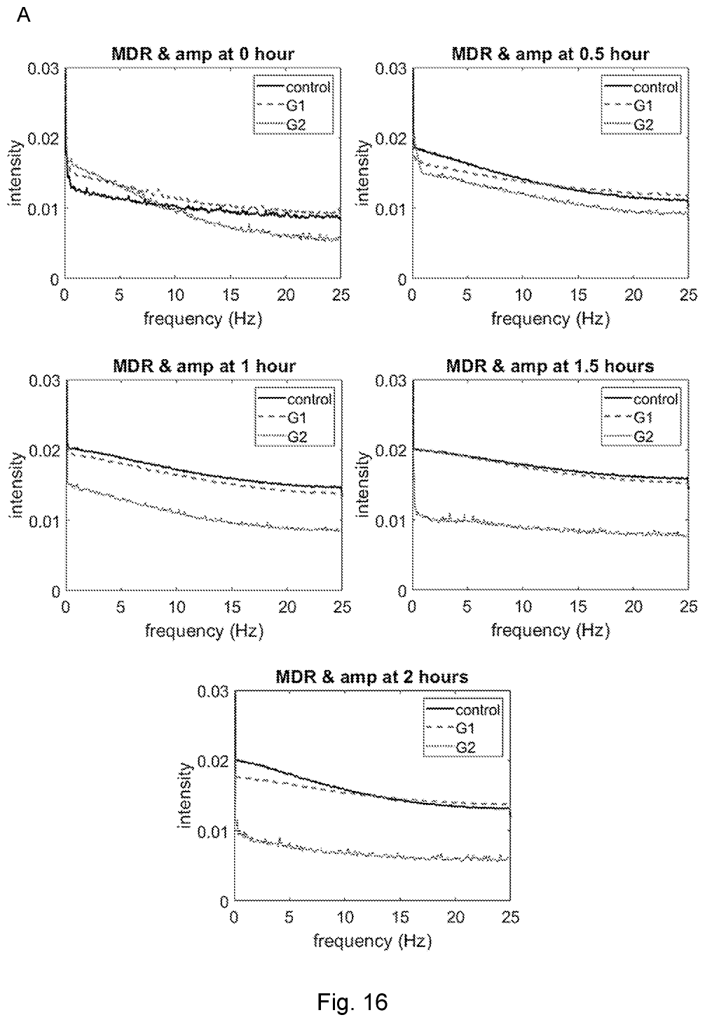

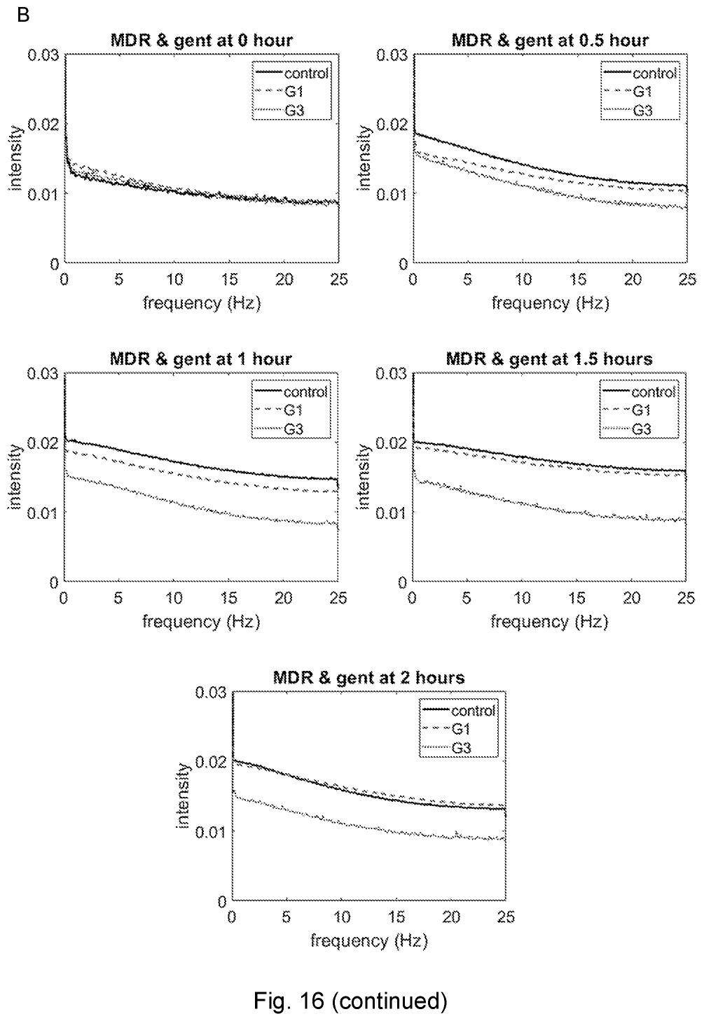

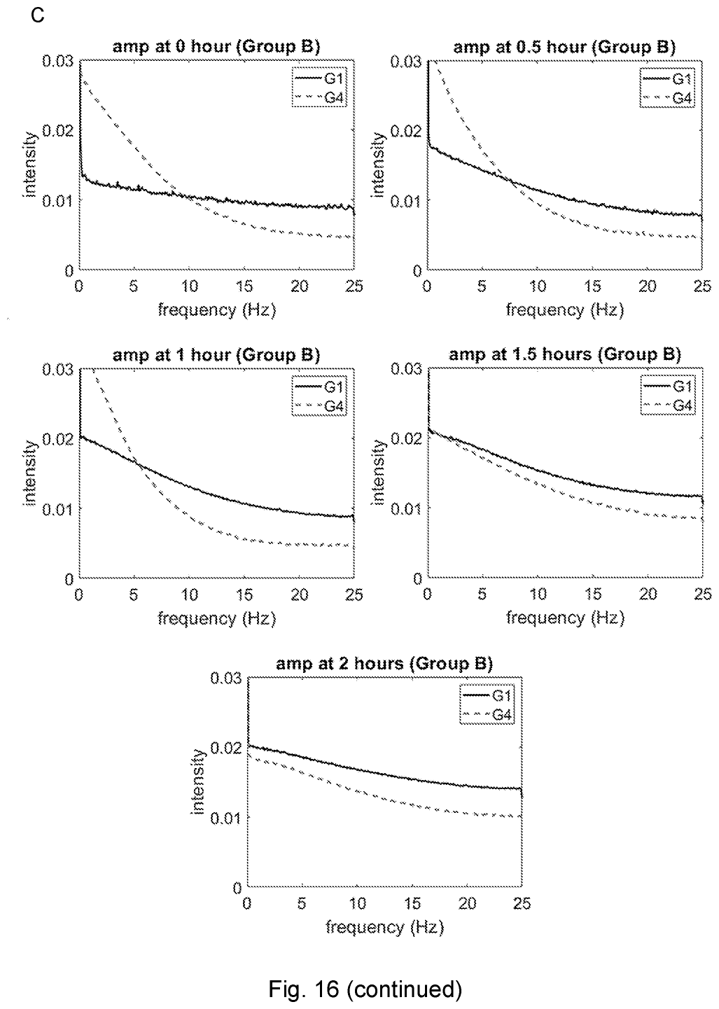

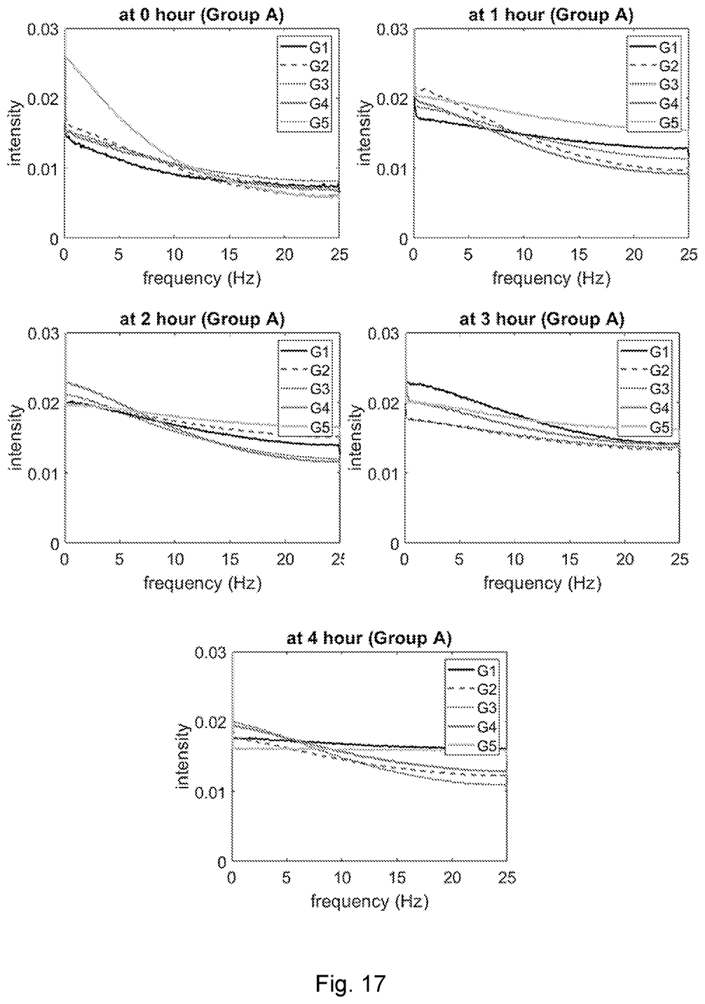

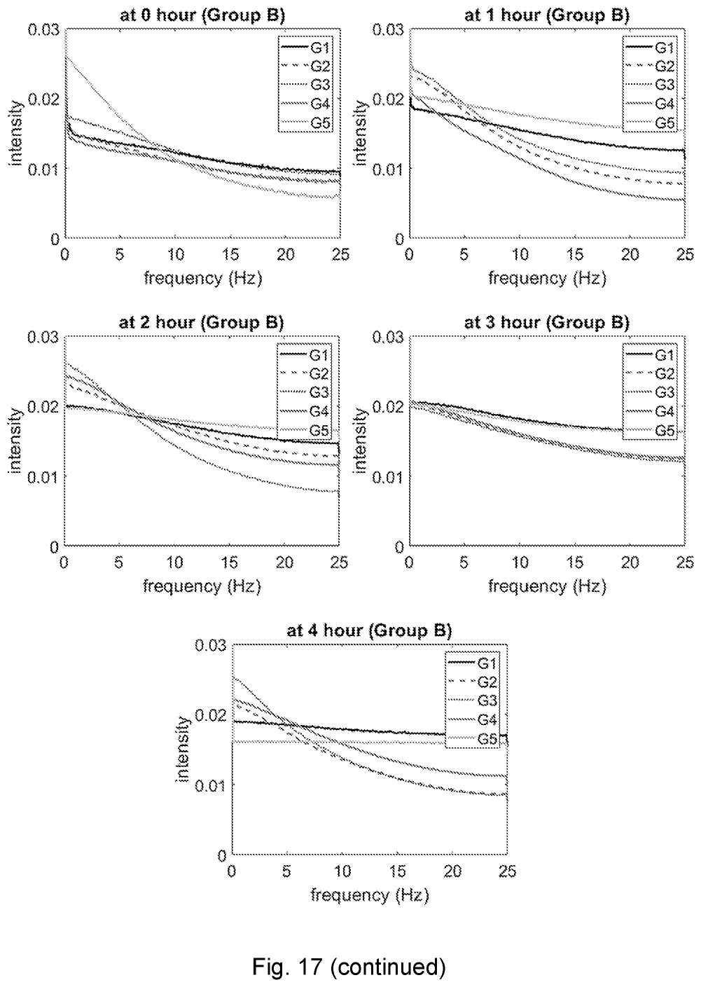

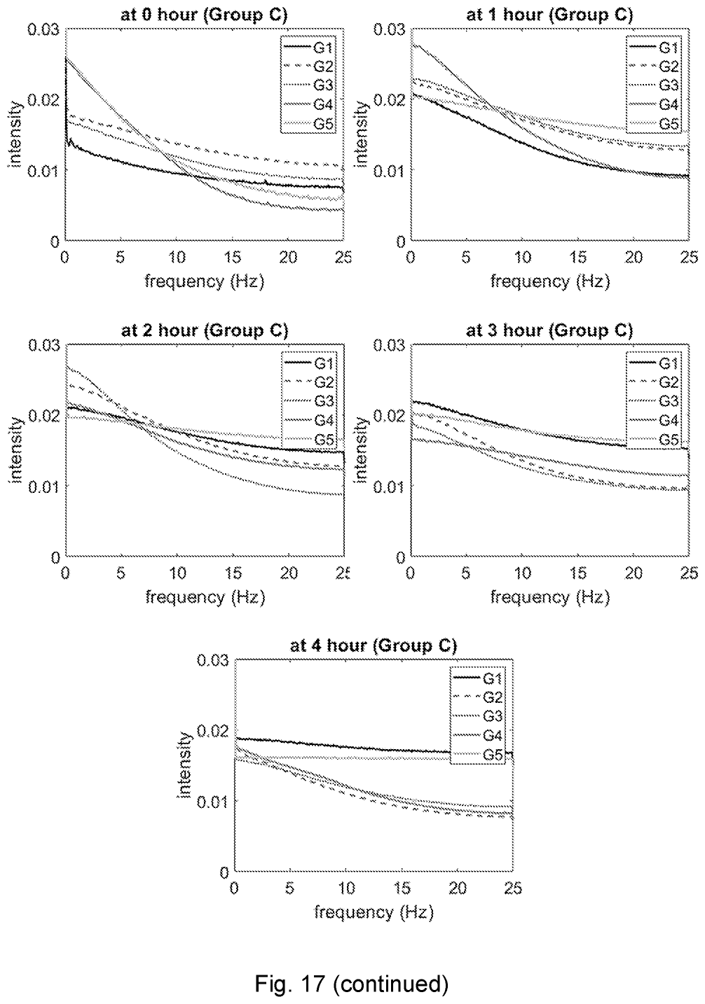

The DC frequency component is normalized to 1 for each individual pixel after the FFT operations. For 100×200 resizing factor, we have 20,000 samples for each group. The average FFT curve is calculated and plotted in FIG. 16. In our experiments, the bandwidth up to 25 Hz was used. After 1.5 hours, the 0.25× and 0.5×MIC groups (resistant groups), have more high frequency contributions compared to the 1× and 2×MIC groups (susceptible groups). This indicates that resistant groups exhibit more active motion than the susceptible groups. However, the difference between resistant and susceptible groups was not clearly distinguished at 0.5 hour, and 1 hour based on the averaged curves. The FT analysis indicate that, it contains information about bacterial motion and provides features that machine learning algorithms can analyze and use for predictions of antibiotic susceptibility. Data obtained using the gentamicin group were processed with the same analysis methods as for ampicillin. As shown in FIG. 15, 0.25× and 0.5×MIC show higher frequency component. Similar to the ampicillin results, the variance of individual pixel spectra is significant. We utilized machine learning to make pixel-level predictions and then apply a voting strategy for prediction of MIC and susceptibility.

### Estimating Size

Using the optimized DLSI setup, we can also extract the angular dependence of the speckle intensity profile (see “Mie scattering analysis”). By fitting the simulated Mie scattering model parameters with experimentally measured speckle images, we can estimate the particle size (Tables 2 and 3). Using this method, we can monitor the change of particle/bacteria size over time with different antibiotic concentrations. More interestingly, our results show that change of bacterial size over time is much more significant with ampicillin treatment compared to gentamicin. We believe this is related to different action mechanisms for ampicillin and gentamicin, with the former targeting cell wall synthesis which is more directly relevant to size of cells, while gentamicin's main target is protein synthesis.

### Confusion Matrix for the ANN Model and Time-Evolution

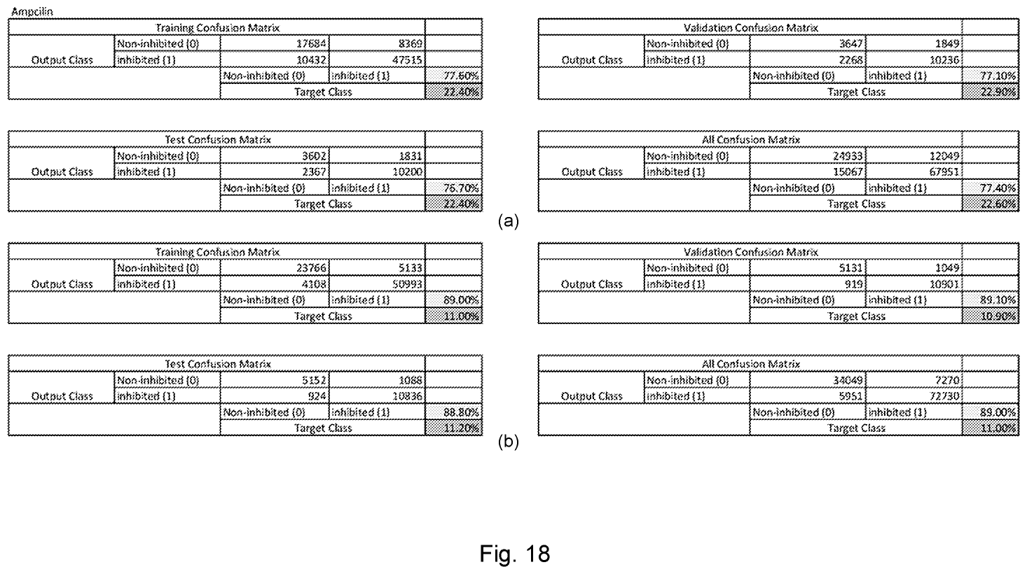

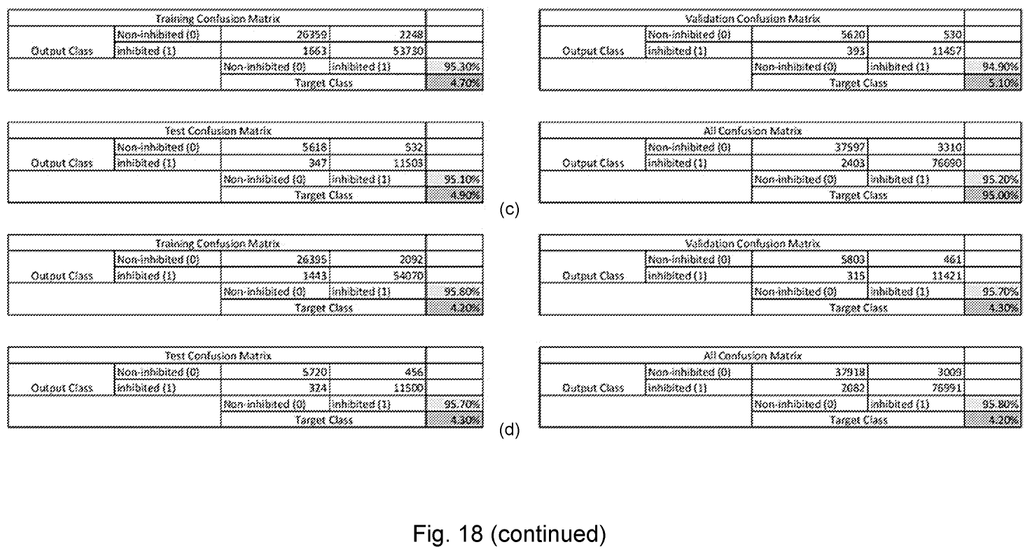

In the confusion matrix of FIG. 18, the lighter-shaded box is the percentage of the correct pixel-level prediction, while the darker-shaded box indicates the incorrect pixel-level prediction. The training has early stopping when the validation error deviate from the training error, the learning algorithm stop and save the parameter values. For t₁, the overall accuracy is 77.4%. Compared to the result of testing on the independent experiments, the algorithm can not make accurate prediction. However, at t₂, t₃, t₄, the prediction accuracy is 89%, 95.2%, 95.8% respectively. And we observe correct predictions for independent experiments.

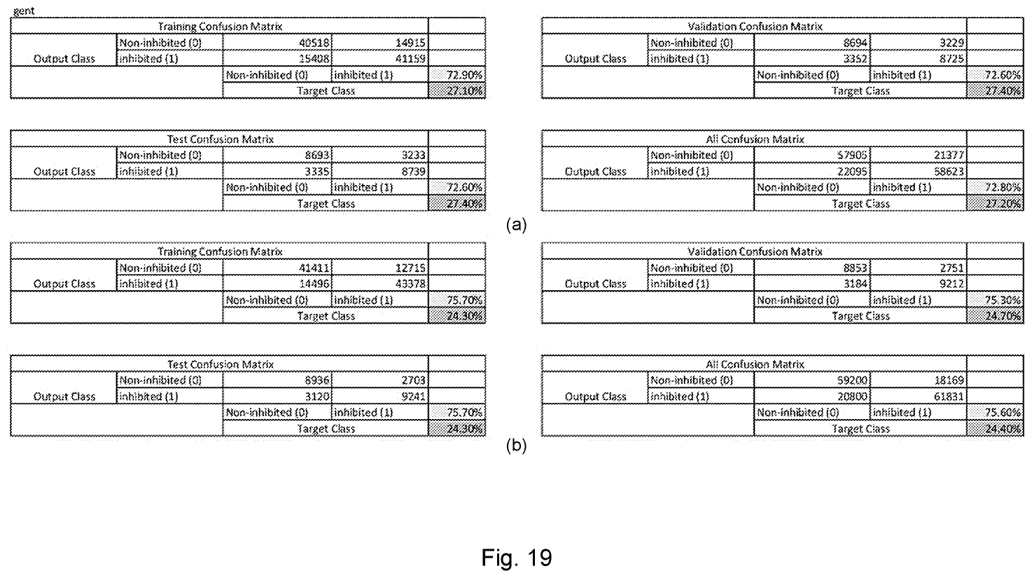

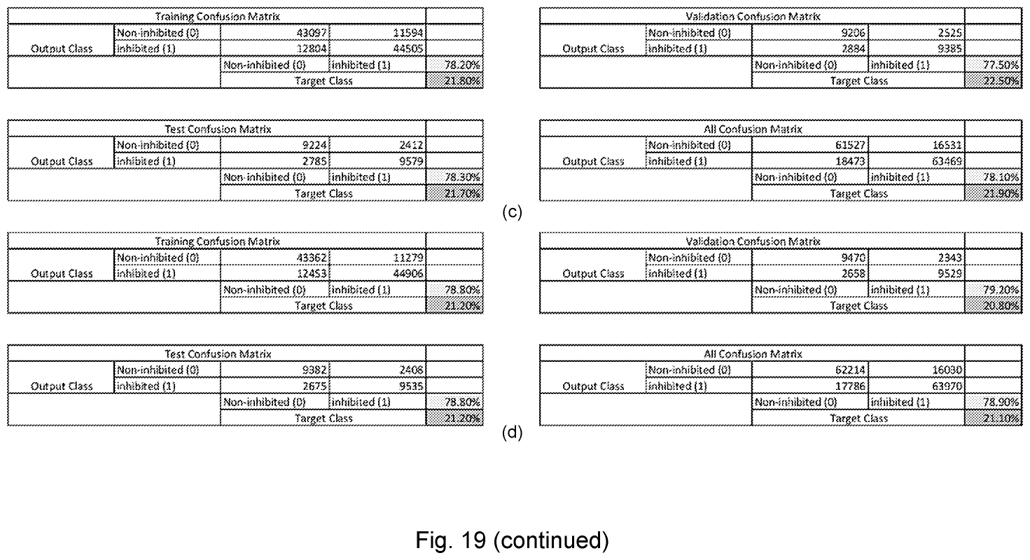

In the confusion matrix of FIG. 19, the lighter-shaded box is the percentage of the correct pixel-level prediction, while darker-shaded box indicates the incorrect pixel-level prediction. The training has early stopping when the validation error deviates from the training error, and the learning algorithm stop and save the parameter values. The accuracy increases from t₁ of 72.8% to t₄ of 78.9%.

Although the present disclosure has been described with respect to one or more particular embodiments, it will be understood that other embodiments of the present disclosure may be made without departing from the spirit and scope of the present disclosure.

1. A method for antibacterial susceptibility testing of a sample, comprising:
preparing a set of two or more samples, each sample including a plurality of bacterial cells from a patient;
adding a different amount of a selected drug to each sample of the set of two or more samples;
illuminating at least a portion of a sample of the set of two or more samples using a coherent illumination source;
capturing a series of speckle images over time of at least a portion of the illuminated sample;
determining an inhibition status of the sample using a machine-learning classifier applied to the series of speckle images; and
repeating the steps of illuminating at least a portion of the sample, capturing a series of speckle images over time, and determining an inhibition status of the sample, for each remaining sample of the set of two or more samples.

2. The method of claim 1, further comprising transforming the series of speckle images to a frequency series of speckle images; and wherein determining the inhibition status of the sample uses the machine-learning classifier applied to the frequency series of speckle images.

3. The method of claim 2, wherein the inhibition status of the sample is determined using the machine-learning classifier by:
classifying each pixel of the frequency series of speckle images as either inhibited or not inhibited using the machine-learning classifier;
calculating a percentage of pixels classified as inhibited; and
determining an inhibition status of inhibited when the percentage of pixels classified as inhibited is greater than 50%, and an inhibition status of not-inhibited when the percentage of pixels classified as inhibited is less than or equal to 50%.

4. The method of claim 3, further comprising normalizing the frequency series of speckle images such that a DC term is normalized to 1.

5. The method of claim 2, wherein the machine-learning classifier is an artificial neural network having a number of neurons in an input layer corresponding to a number of frequency components in the frequency series of speckle images.

6. The method of claim 1, wherein the amounts of the selected drug added to each sample of the set of two or more samples yields concentrations of the selected drug that are different percentages of a minimum inhibitory concentration (MIC).

7. The method of claim 1, wherein the series of speckle images over time is captured at multiple time points after adding the selected drug to the samples.

8. The method of claim 1, wherein the series of speckle images over time is captured at a time at least 60 minutes after adding the selected drug to the samples.

9. The method of claim 1, wherein a minimum inhibitory concentration (MIC) of the selected drug is determined based on the inhibition status of each sample.

10. The method of claim 1, wherein the series of speckle images is captured in an angular range defined by a Mie scattering model, between an optical axis of the image sensor and an optical axis of the illumination source.

11. The method of claim 10, further comprising determining an average bacterium size of each sample using the Mie scattering model fitted with the series of speckle images.

12. The method of claim 1, further comprising determining an average intensity value of each series of speckle images.

13. A system for antibacterial susceptibility testing of a sample, comprising:
a sample holder;
a coherent illumination source configured to illuminate at least a portion of a sample within the sample holder;
an image sensor positioned to receive light scattered by the sample thereby creating a series of speckle images over time;
a processor, wherein the processor is configured to:
receive from the image sensor the time series of speckle images;
determine an inhibition status of the sample using a machine-learning classifier applied to the series of speckle images over time.

14. The system of claim 13, wherein the processor is further configured to transform the series of speckle images over time into a frequency series of speckle images, and wherein determining the inhibition status of the sample uses the machine-learning classifier applied to the frequency series of speckle images.

15. The system of claim 14, wherein the processor is configured to determine an inhibition status of the test sample using the machine-learning classifier by:
classifying each pixel of the frequency series of images as either inhibited or not inhibited using the machine-learning classifier;
calculating a percentage of pixels classified as inhibited; and
determining an inhibition status of inhibited when the percentage of pixels classified as inhibited is greater than 50%, and an inhibition status of not-inhibited when the percentage of pixels classified as inhibited is less than or equal to 50%.

16. The system of claim 15, wherein the frequency series of speckle images is normalized such that a DC term is normalized to 1.

17. The system of claim 14, wherein the machine-learning classifier is an artificial neural network having a number of neurons in an input layer corresponding to a number of frequency components in the frequency series of speckle images.

18. The system of claim 13, wherein an optical axis of the image sensor is positioned in an angular range with respect to an optical axis of the illumination source defined by a Mie scattering model.

19. The system of claim 18, wherein the processor is further configured to determine an average bacterium size of the sample using the Mie scattering model fitted with the series of speckle images.

20. The system of claim 13, wherein the series of speckle images is captured over a measurement period of 10 seconds.

21. The system of claim 13, wherein the processor is further configured to resize the series of speckle images.

22. The system of claim 13, wherein the processor is further configured to determine an average intensity value of the series of speckle images.

23. The system of claim 13, wherein the image sensor is a complementary metal-oxide semiconductor (CMOS) camera.
